(19) United States
(12) Patent Application Publication
(10) Pub. No.: US 2017/0119719 A1
(43) Pub. Date: May 4, 2017

(54) ADMINISTRATION OF INGENOL MEBUTATE

(71) Applicant: LEO Laboratories Limited, Dublin (IE)

(72) Inventor: Steven Martin Ogbourne, Pinbarren (AU)

(21) Appl. No.: 15/351,042

(22) Filed: Nov. 14, 2016

Related U.S. Application Data
(63) Continuation of application No. 14/195,842, filed on Mar. 3, 2014, now abandoned.
Application No. 14/195,842 is a continuation of application No. 13/747,474, filed on Jan. 22, 2013, now abandoned.

(51) Int. Cl. A61K 31/22; A61K 9/00; A61K 9/06

(52) U.S. Cl. CPC A61K 31/22; A61K 9/06; A61K 9/0014

(57) ABSTRACT

The present invention relates to novel crystalline forms of ingenol ingenol-3-angelate and methods of preparation and use thereof. More specifically, the invention relates to a novel crystalline form and purified forms of the compound of Formula 1 (ingenol-3-mebutate; ingenol-3-angelate; isoform ‘b’; PEP005), which is characterized by, for example, attenuated total reflectance Fourier transform infrared (FTIR-ATR) spectroscopy, single crystal X-Ray crystallography (XRC), X-ray powder diffraction, and, Differential Scanning calorimetry (DSC), and methods of preparation and use thereof.

### RELATED APPLICATIONS

This application is a continuation of U.S. patent application Ser. No. 13/747,474, filed Jan. 22, 2013, pending. This application and U.S. patent application Ser. No. 13/747,474 are also related to U.S. patent application Ser. No. 13/747,474 application Ser. No. 13/088,910, filed Apr. 18, 2011, which claims the benefit of and priority to U.S. Provisional Patent Application No. 61/325,032, filed Apr. 16, 2010. The contents of each of the foregoing applications are incorporated herein by reference in their entirety.

### FIELD OF THE INVENTION

This invention relates to a novel crystalline form of ingenol mebutate (ingenol-3-angelate), to purified and/or isolated ingenol-3-angelate, to methods of preparation thereof, and to uses thereof.

### BACKGROUND OF THE INVENTION

Ingenol mebutate has the structure shown in Formula 1 and the following chemical names: 2S
1) 2-Butenoic acid, 2-methyl (1aR,5R,5aS,6S,8aS,9R,10aR)-1a,2,5,5a,6,9,10,10a-octahydro-5,5a-dihydroxy-4-(hydroxymethyl)-1,1,7,9-tetramethyl-11-oxo-1H-2,8a-methanocyclopenta[a]cyclopropa[e]cyclodecen-6-yl ester, (2Z)—
2) (1aR,2S,5R,5aS,6S,8aS,9R,10aR)-5,5a-dihydroxy-4-(hydroxy-methyl)-1,1,7,9-tetramethyl-11-oxo-1a,2,5,5a,6,9,10,10a-octahydro-1H2,8a-methanocyclopenta[a]cyclopropa[e]cyclodecen-6-yl(2Z)-2-methylbut-2-enoate)

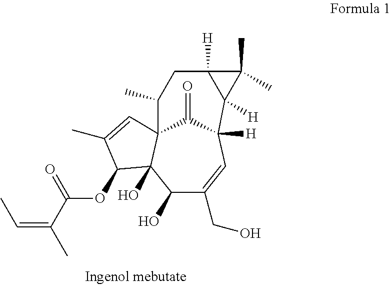

Ingenol mebutate (synonyms: PEP005, ingenol-3-angelate, CAS no. 75567-37-2) can be isolated from various Euphorbia species, and particularly from Euphorbia peplus and Euphorbia drummondii. Ingenol mebutate is believed to exists in three isomeric forms; ingenol-3-mebutate (isoform ‘b’; PEP005), ingenol-5-mebutate (isoform ‘a’; PEP015) and ingenol-20-mebutate (isoform ‘c’; PEP025). Ingenol-3-mebutate (Isoform ‘b’; PEP005) has the structure shown in Formula 1.

The preparation of Ingenol mebutate by extraction with 95% ethanol from the sap of Euphorbia peplus, Euphorbia hirta and/or Euphorbia drummondi; followed by chromatographic purification has been disclosed in EP1015413 B1, which is incorporated herein by reference in its entirety. Isolation from Euphorbia peplus has also been described by Hohmann et. al. Planta Med. 66, 3, (2000), which is incorporated herein by reference in its entirety. Other patent applications directed to ingenol mebutate and other pharmaceutically active ingenol derivatives include WO 2008/131491, WO 2007/068963, WO 2007/059584 and WO 2007/053912, each of which is incorporated herein by reference in its entirety.

Ingenol mebutate has been found to be highly toxic for skin cancer cells via rapid mitochondrial disruption and cell death by primary necrosis, whereas normal cells are less sensitive to ingenol mebutate.

Ingenol mebutate has been shown to be a potent anti-cancer drug and therapeutically effective in microgram quantities. Recent findings from a Phase III study evaluating ingenol mebutate in the treatment of actinic keratosis (AK), a common pre-cursor to skin cancer, were presented at the 68th Annual Meeting of the American Academy of Dermatology (AAD) (Scientific Session Poster Discussion: P105). Results from REGION-I of the study demonstrated that treatment with ingenol mebutate Gel once daily for 2 consecutive days (n=117) on non-head locations resulted in significant clearance of AK lesions when compared with the vehicle or placebo (n=118). The study showed a median reduction of about 66.7% in the number of AK lesions, (p<0.0001), a complete clearance rate of about 27.4% (p<0.0001) including on the extremely difficult-to-treat back of hand and arm locations, and a partial clearance rate of about 44.4% (p<0.0001).

Ingenol mebutate is commercially available in amorphous form, i.e., from Sigma-Aldrich.

### SUMMARY OF THE INVENTION

This invention is directed to a novel crystalline form of ingenol mebutate, to pharmaceutical compositions comprising this crystalline form, and to methods of its preparation and use.

In certain embodiments, the invention provides crystalline ingenol mebutate.

In certain embodiments, crystalline ingenol mebutate is not a solvate.

In certain embodiments, crystalline ingenol mebutate is orthorhombic.

In certain embodiments, crystalline ingenol mebutate is obtained from acetonitirile or a mixture of ethanol and water.

A first embodiment of the invention comprises a crystalline form of the compound of Formula 1 (isoform b; ingenol-3-mebutate; ingenol-3-angelate; PEP005).

In one embodiment, a crystalline form of the compound of Formula 1 is characterized by an attenuated total reflectance fourier transform infrared (FTIR-ATR) spectrum substantially as shown in FIG. 1. FIG. 1 shows graph 1, which is a characteristic FTIR-ATR spectrum of a crystalline form of the compound of Formula 1 acquired using the Universal Attenuated Total Reflectance accessory on a Perkin Elmer Spectrum One FTIR spectrometer.

In another embodiment, a crystalline form of the compound of Formula 1 is characterized by an attenuated total reflectance fourier transform infrared (FTIR-ATR) spectrum exhibiting one or more attenuated total reflectance peaks at approximately 3535, 2951, 1712, 1456, 1378, 1246, 1133, 1028 and/or 956 cm⁻¹ (±3 cm⁻¹), respectively.

In yet another embodiment, a crystalline form of the compound of Formula 1 is characterized by one or more single crystal parameters substantially as shown in Table 1:

TABLE 1
Crystal Parameters

| | |
|---|---|
| Crystal system | Orthorhombic |
| Space Group: | P2₁2₁2₁ |
| Unit Cell Dimensions: | a = 7.1295(4) A |
| | b = 7.7558(4) A |
| | c = 41.375(2) A |
| Volume: | 2287.9(2) A³ |
| Molecules per Unit Cell (Z) | 4 |
| Density (calculated) | 1.250 Mg/m³ |

In yet another embodiment, a crystalline form of the compound of Formula 1 is characterized by atoms at atomic positions relative to the origin of the unit cell substantially as shown in Table 2, or bond lengths or bond angles substantially as shown in Table 3 (infra Example 3).

In yet another embodiment, a crystalline form of the compound of Formula 1 is characterized by a structure obtained by single crystal X-Ray crystallography (XRC) substantially as shown in FIG. 2. FIG. 2 shows a configuration of a crystalline form of the compound of Formula 1 obtained by single crystal X-Ray crystallography (XRC). The thermal ellipsoids were drawn at the 35% probability level.

In yet another embodiment, a crystalline form of the compound of Formula 1 is characterized by the crystal data and structure refinements substantially as shown in FIG. 3.

In yet another embodiment, a crystalline form of the compound of Formula 1 is characterized by X-ray powder diffraction peaks substantially as shown in FIG. 4.

In yet another embodiment, a crystalline form of the compound of Formula 1 is characterized by X-ray powder diffraction peaks at an angle of refraction of about 4.3°, about 8.5° and about 13.0° 20.

In yet another embodiment, a crystalline form of the compound of Formula 1 is characterized by a Raman spectrum substantially as shown in FIG. 5.

In yet another embodiment, a crystalline form of the compound of Formula 1 is characterized by a differential scanning calorimetry (DSC) curve substantially as shown in FIG. 6.

In yet another embodiment, a crystalline form of the compound of Formula 1 is characterized by a DSC curve comprising an event with an onset at about 153° C. (±5° C.). The characteristic DSC curve was characterized using a Perkin Elmer DSC 8500, with a heating rate of 20° C./min, and the average enthalpy was about 85±about 8 mJ/mg.

In yet another embodiment, a crystalline form of the compound of Formula 1 is characterized by a thermogravimetric (TG) curve substantially as shown in FIG. 7.

In yet another embodiment, a crystalline form of the compound of Formula 1 is obtainable by crystallization of the compound of Formula 1 from a solvent such as acetone, acetonitrile, ethanol, 2-propanol, heptane, methyl tert-butyl ether, monoglyme, toluene, a mixture of acetone and heptane, a mixture of acetone and c-hexane, a mixture of acetone and i-octane, a mixture of acetone and xylene, a mixture of acetonitrile and water, a mixture of ethanol and water, a mixture of 2-propanol and water, a mixture of 2-propanol and heptane, a mixture of 1,4-dioxane and heptane, a mixture of 1,4-dioxane, dimethyl sulfoxide and heptane, or a mixture of toluene and heptane. In certain embodiments, crystalline ingenol mebutate is obtained from acetonitrile or from a mixture of ethanol and water.

Preferably, a crystalline ingenol mebutate or isolated or purified ingenol mebutate of the present invention has a crystalline purity of at least about 99.5% (e.g., as measured by HPLC as described in Example 1). In certain embodiments, the crystalline purity is at least about 99.7% or more preferably about 99.72%. In certain embodiments, the crystalline purity is at least about 99.9%.

The invention also provides isolated or purified ingenol mebutate, crystalline ingenol mebutate, or highly pure crystalline ingenol mebutate, e.g., for use as a medicament. The present invention also contemplates the use of crystalline ingenol mebutate or highly pure crystalline ingenol mebutate for the topical treatment of skin disorders, including skin cancers and other skin conditions involving neoplastic cells, such as solar keratosis or actinic keratosis. The skin cancers contemplated by the present invention include, amongst others, melanoma, malignant melanoma, merkel cell carcinoma, squamous cell carcinoma, and basal cell carcinoma (BCC), including superficial-basal cell carcinoma (sBCC).

The invention also provides a pharmaceutical composition comprising crystalline ingenol mebutate or highly pure ingenol mebutate, and one or more pharmaceutically acceptable carriers or vehicles. In certain embodiments, the pharmaceutical composition is suitable for topical administration of a pharmaceutical composition to deliver an effective amount of crystalline ingenol mebutate to a treatment area of the skin to treat a skin disorder. In accordance with the present invention, the pharmaceutical composition can be formulated as a liquid or semi-solid, such as a gel, cream, ointment, salve, balm, liquid, suspension or lotion. The invention also provides a method of making a pharmaceutical composition comprising crystalline ingenol mebutate, the method comprising combining crystalline ingenol mebutate with a pharmaceutically acceptable carrier or vehicle.

The present invention also provides a method of treating a skin disorders, such as skin cancer or other skin conditions involving neoplastic cells, such as actinic keratosis or solar keratosis. The method comprises applying an effective amount of a pharmaceutical composition of the invention to a treatment area on a subject in need thereof.

### DEFINITIONS

The term “C₁-C₆ linear or branched alkyl alcohols” includes methanol, ethanol, 1-propanol, 2-propanol, 1-butanol, tert-butanol, and 2-butanol.

The term “C₂-C₆ linear or branched alkyl nitriles” includes acetonitrile and propionitrile.

### BRIEF DESCRIPTION OF THE DRAWINGS

FIG. 1 shows a characteristic FTIR-ATR spectrum of a crystalline form (crystalline Form A) of the compound of Formula 1, acquired using the Universal Attenuated Total Reflectance accessory on a Perkin Elmer Spectrum One FTIR spectrometer.

FIG. 2 shows a configuration of a crystalline form (crystalline Form A) of the compound of Formula 1, obtained by single crystal X-Ray crystallography (XRC). The thermal ellipsoids were drawn at the 35% probability level.

FIG. 3 shows a configuration of a crystalline form (crystalline Form A) of the compound of Formula 1, according to the crystal data and structure refinements of Table 4.

FIG. 4 shows an XRPD diffractogram of a crystalline form (crystalline Form A) of the compound of Formula 1. Characteristic peaks of the compound of Formula 1 in the XRPD diffractogram are present at 4.3°, 8.5° and 13.0° 20.

FIG. 5 shows a Raman spectrum of a crystalline form (crystalline Form A) of the compound of Formula 1.

FIG. 6 shows a DSC curve of a crystalline form (crystalline Form A) of the compound of Formula 1.

FIG. 7 shows a TG curve of a crystalline form (crystalline Form A) of the compound of Formula 1.

FIG. 8 shows an XRPD diffractogram of a crystalline ingenol mebutate form X. Characteristic peaks of form X in the XRPD diffractogram are present at 3.9° and 5.5° 2θ.

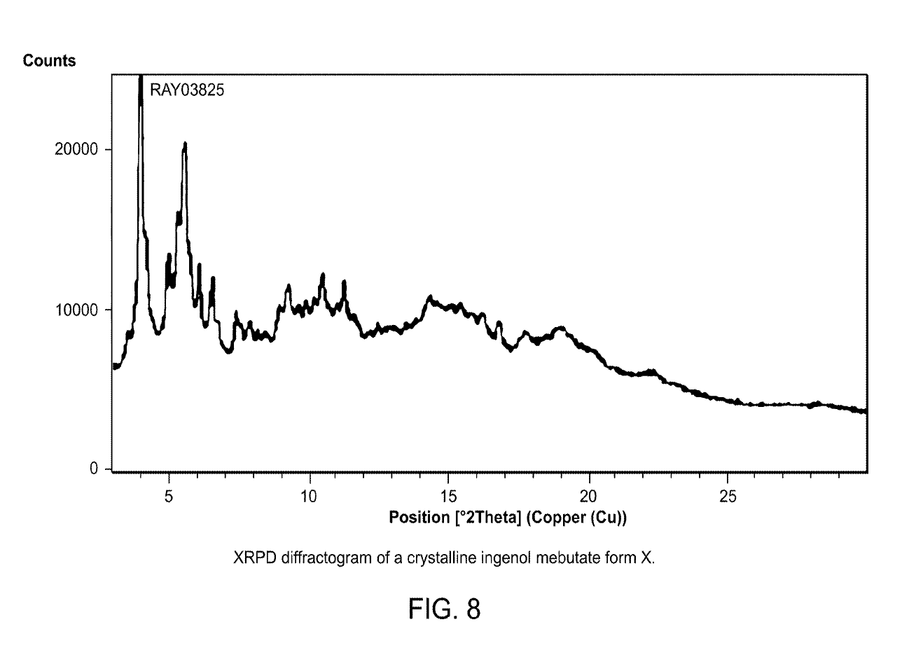

FIG. 9 shows a DSC curve of a crystalline ingenol mebutate form X.

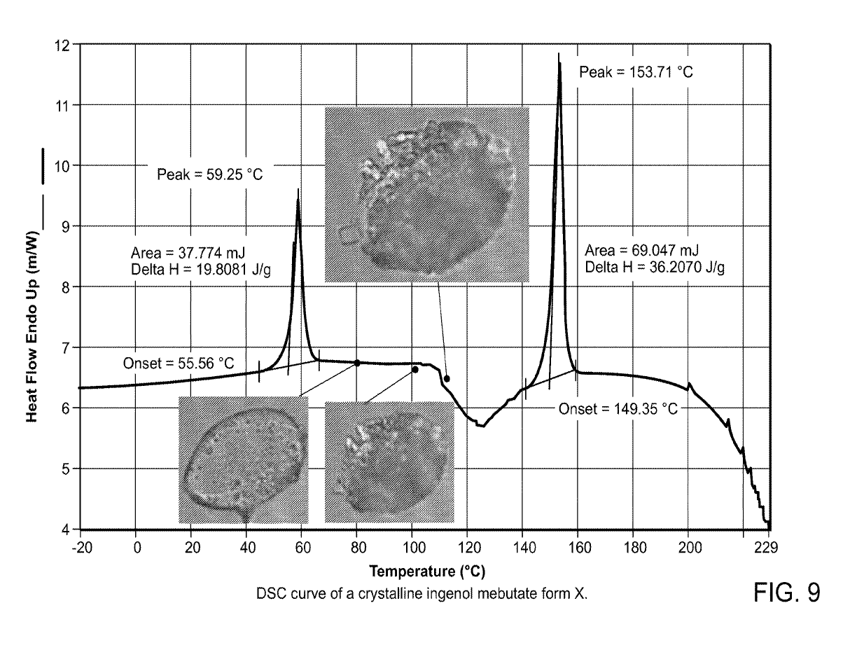

FIG. 10 shows a TG curve of a crystalline ingenol mebutate form X.

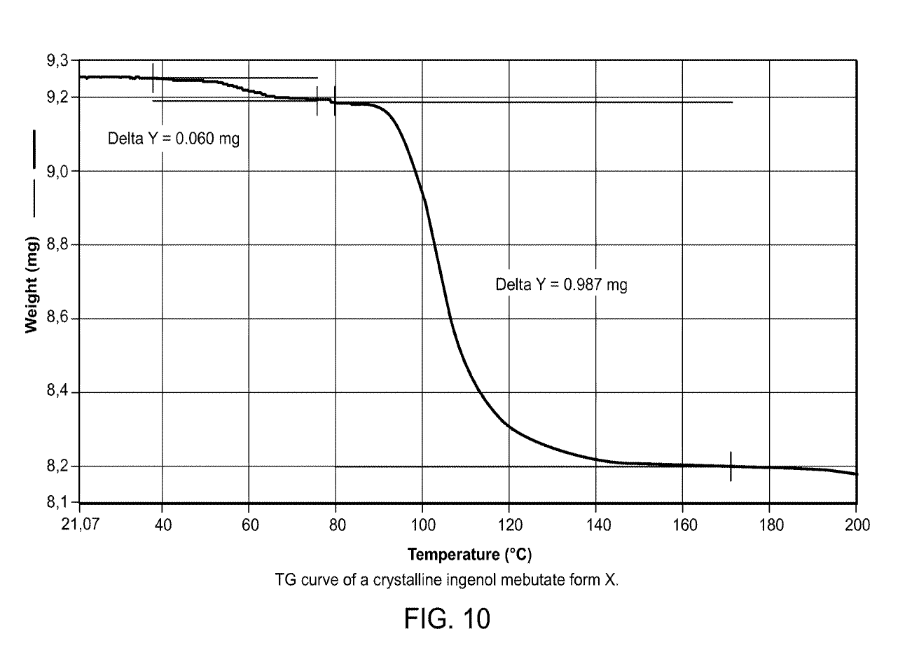

FIG. 11 shows an XRPD diffractogram of a crystalline ingenol mebutate form Y. Characteristic peaks of form Y in the XRPD diffractogram are present at 5.1°, 6.3° and 10.7° 2θ.

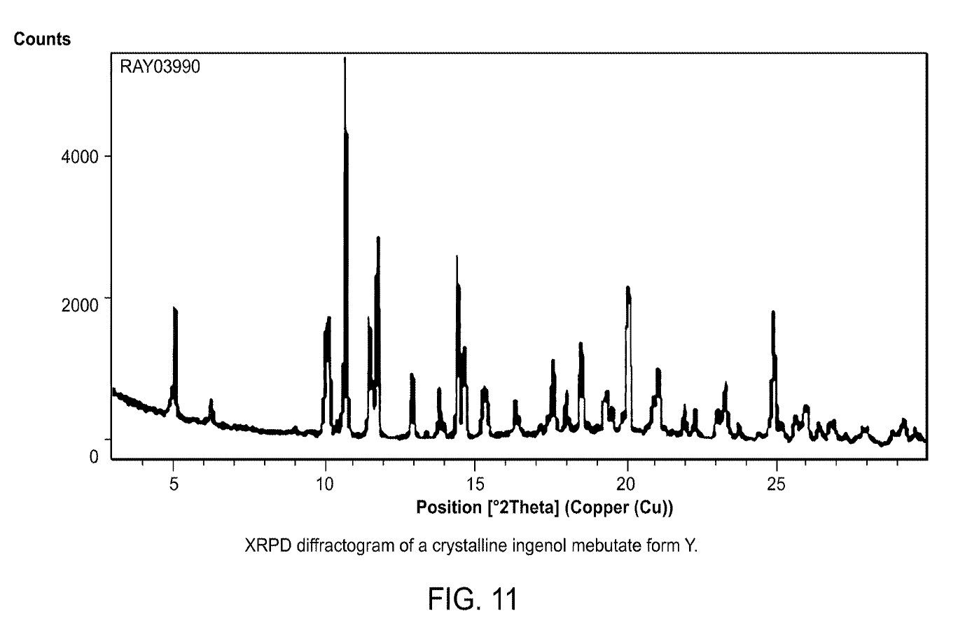

FIG. 12 shows a DSC curve of a crystalline ingenol mebutate form Y.

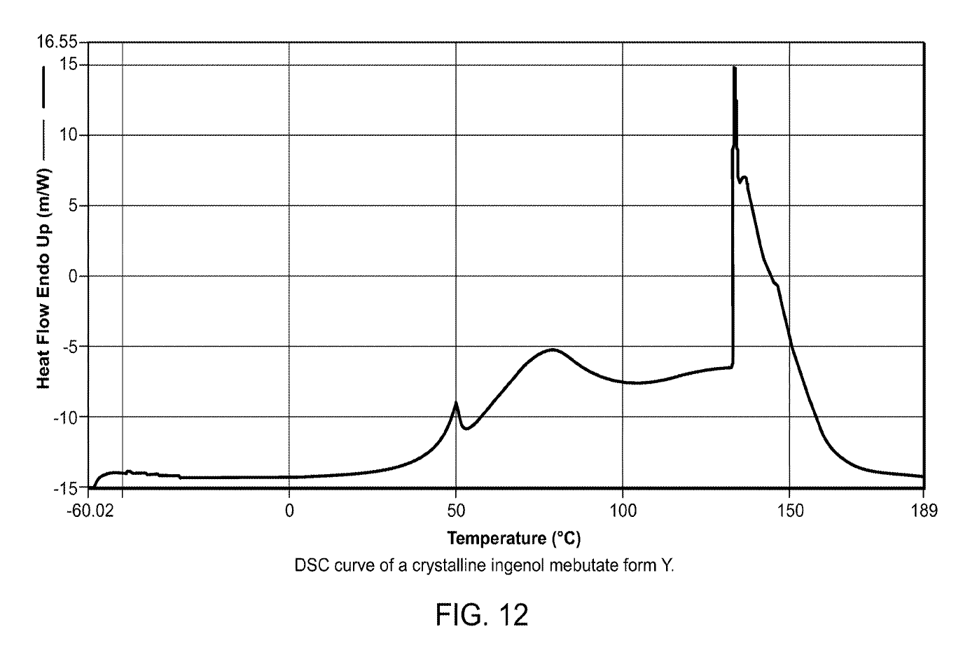

FIG. 13 shows XRPD diffractograms of crystalline ingenol mebutate forms.

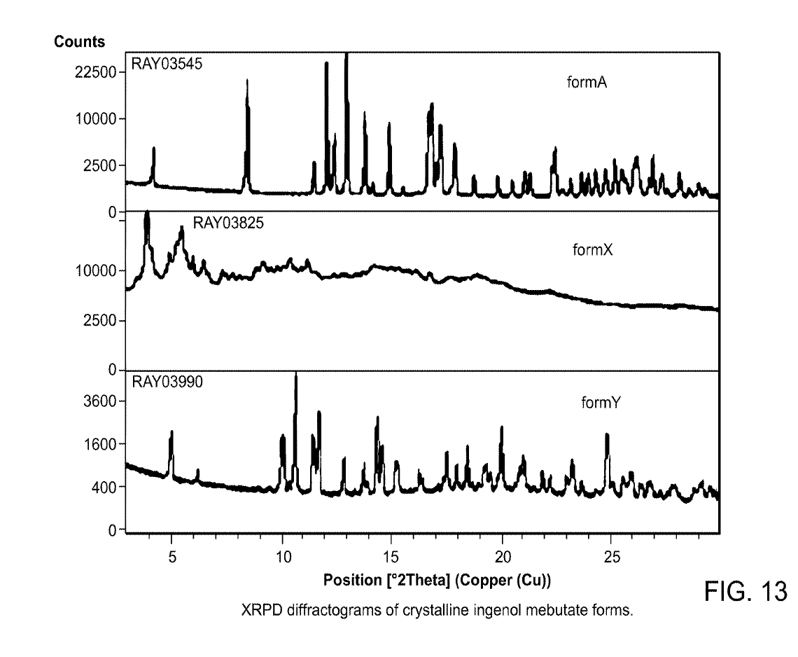

FIG. 14 shows an XRPD diffractogram of amorphous ingenol mebutate.

FIG. 15 shows a DSC curve of amorphous ingenol mebutate.

FIG. 16 shows a DSC curve of amorphous ingenol mebutate with temperature cycling.

FIG. 17 shows an XRPD diffractogram of crystalline ingenol mebutate after 24H slurry.

FIG. 18 shows XRPD diffractograms of crystalline ingenol mebutate after milling.

FIG. 19 shows absorption of ingenol-3-mebutate (PEP005) through rat, human and mini-pig dermatone membranes.

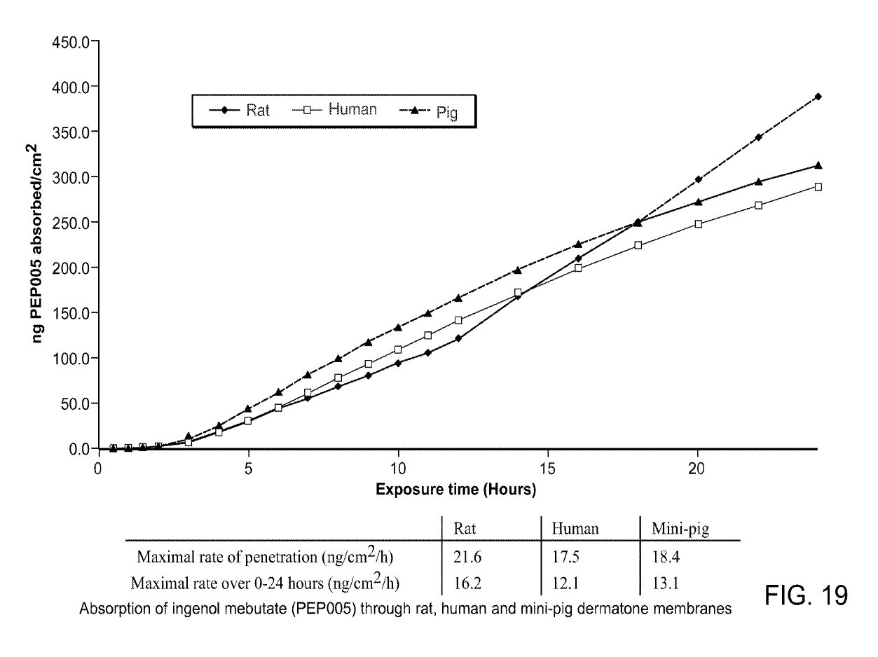

### DETAILED DESCRIPTION OF THE INVENTION

This present invention relates to novel crystalline forms of ingenol mebutate. The crystalline form of the compound of Formula 1 surprisingly possesses enhanced chemical stability, particularly when compared to amorphous ingenol mebutate and other crystalline forms thereof, and physical properties which facilitate the handling and manufacture of the active pharmaceutical ingredient (API) and finished dosage forms.

The ease and safety with which dosage forms are prepared, as well as the properties of the drug, may depend on factors such as, but not limited to, the stability, purity, solubility, homogeneity, hygroscopicity, and flow characteristics of the API. These properties may be altered or improved if a specific crystalline, rather than an amorphous, form of the API can be produced. In accordance with the present invention, it has been surprisingly discovered that the processability and physicochemical properties of a crystalline igenol mebutate are advantageous. For pharmaceutical formulations of the present invention, the availability of crystalline igenol mebutate uniquely provides for a range of topical formulations, including for example, suspensions or micronisation or nano-processing techniques. The process for obtaining the crystalline form of the compound of formula I additionally improves the stability and purity of the compound and eliminates byproducts from the previous isolations steps.

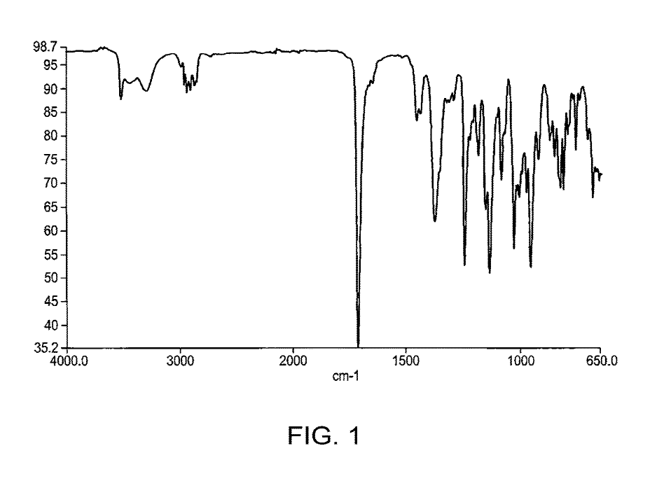

Graph 1 of FIG. 1 shows a characteristic FTIR-ATR spectrum of a crystalline form of the compound of Formula 1.

Table 1 above shows the Single Crystal Parameters for a crystalline form of the compound of Formula 1. Selected atomic coordinates and isotropic thermal parameters determined from the data are provided in Table 2. Bond lengths and bond angles are set forth in Table 3. Other crystal data and structure refinement details are provided in Table 4.

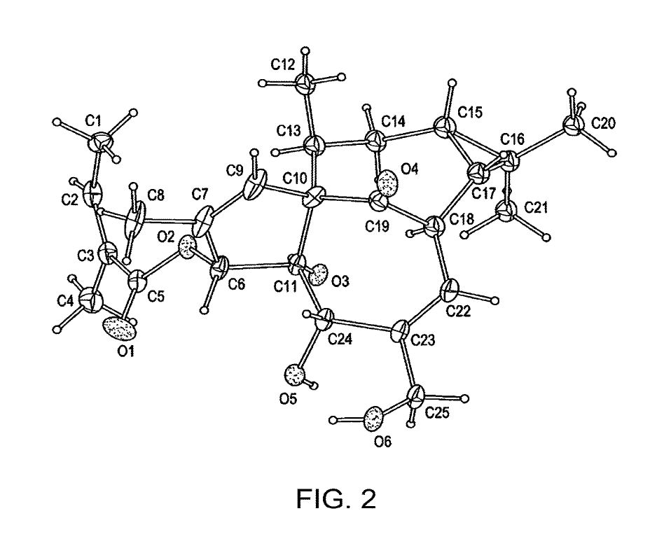

FIG. 2 shows the Single Crystal Configuration of a crystalline form of the compound of Formula 1.

A crystalline composition of matter disclosed herein may be prepared from amorphous (i.e. noncrystalline) or impure ingenol mebutate. The preparation of amorphous ingenol mebutate is disclosed by EP1015413 B1, which is incorporated herein by reference in its entirety.

A presently preferred method of forming crystalline ingenol mebutate comprises dissolving the amorphous compound in a solvent or solvent mixture. Presently preferred solvents include C₁-C₆ linear or branched alkyl alcohols such as ethanol and C₂-C₆ linear or branched alkyl nitriles such as acetonitrile. In certain embodiments, the solvent is acetone, acetonitrile, ethanol, 2-propanol, heptane, methyl tert-butyl ether, monoglyme, toluene, a mixture of acetone and heptane, a mixture of acetone and c-hexane, a mixture of acetone and i-octane, a mixture of acetone and xylene, a mixture of acetonitrile and water, a mixture of ethanol and water, a mixture of 2-propanol and water, a mixture of 2 propanol and heptane, a mixture of 1,4-dioxane and heptane, a mixture of 1,4-dioxane, dimethyl sulfoxide and heptane, or a mixture of toluene and heptane. In certain embodiments, crystalline ingenol mebutate is obtained from acetonitrile or from a mixture of ethanol and water.

Preferably, the solvent is heated, the amorphous compound dissolved in it to a point approximately equal to saturation, water optionally added, and the resulting solution allowed to cool to a temperature at which the full amount of the compound dissolved is no longer soluble in the solvent or solvent mixture. Crystals are isolated by filtration and dried, optionally in vacuo at an elevated temperature.

In yet another aspect, the present invention relates to isolated crystalline ingenol mebutate as defined above, which has a polymorphic purity of at least about 80%, such as about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In yet another aspect, the present invention relates to isolated crystalline ingenol mebutate as defined above, which has a degree of crystallinity of at least about 80%, such as about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In yet another aspect, the present invention relates to an isolated crystalline form of the compound of Formula 1 as defined above which contains at least about 90% of isoform ‘b’, i.e. ingenol-3-mebutate, such as about 90%, about about 91%, about 92%, about 93%, about 94%, about 95%, about 96%, about 97%, about 98%, about 99%, or about 100%.

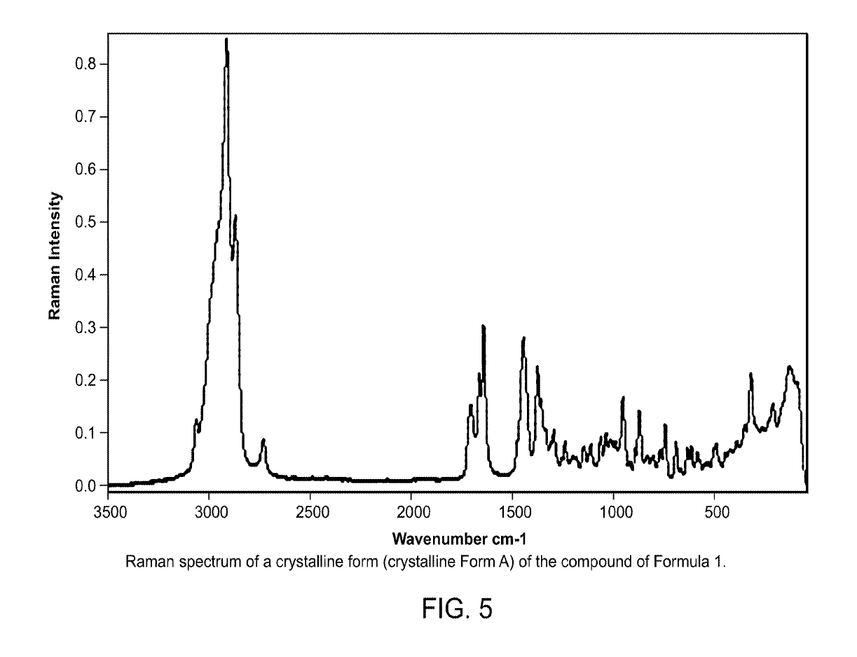

In another aspect, the invention provides crystalline ingenol-3-mebutate. In certain embodiments, the crystalline ingenol-3-mebutate is not a solvate. In certain embodiments, the crystalline ingenol-3-mebutate is characterized by X-ray diffraction peaks at an angle of refraction about 4.3°, about 8.5° and about 13.0° 20. In certain embodiments, the crystalline ingenol-3-mebutate is orthorhombic. In certain embodiments, the crystalline ingenol-3-mebutate belongs to the space group P2₁2₁2₁. In certain embodiments, the crystalline ingenol-3-mebutate is further characterized by an FTIR-ATR spectrum exhibiting attenuated total reflectance peaks at approximately 3535, 2951, 1712, 1456, 1378, 1246, 1133, 1028 and/or 956 cm⁻¹ (±3 cm⁻¹). In certain embodiments, the crystalline ingenol-3-mebutate is further characterized by a differential scanning calorimetry curve comprising an event with an onset at about 153.5±5° C., and the average enthalpy is 85±8 mJ/mg. In certain embodiments, the crystalline ingenol-3-mebutate is obtainable by crystallization of the compound of formula 1 from acetonitrile or a mixture of ethanol and water. In certain embodiments, the crystalline ingenol-3-mebutate is characterized by XRC single crystal parameters that are substantially identical to those provided in Table 1. In certain embodiments, the crystalline ingenol-3-mebutate is characterized by comprising (a) atoms at atomic positions relative to the origin of the unit cell as set forth in Table 2, or (b) bond lengths or bond angles as set forth in Table 3. In certain embodiments, the crystalline ingenol-3-mebutate is characterized by a XRC single crystal structure according to FIG. 2. In certain embodiments, the crystalline ingenol-3-mebutate is characterized by a Raman spectrum substantially as shown in FIG. 5. In certain embodiments, the crystalline ingenol-3-mebutate is characterized by a purity of at least about 99.5%, at least about 99.7%, at least about 99.72%, or at least about 99.9%. In certain embodiments, the crystalline form is rod shape. In certain embodiments, the rod-shape has a size of about 0.3 mm×about 0.05 mm×about 0.05 mm. In certain embodiments, the crystalline form is colorless.

In another aspect, the invention provides crystalline or high-purity ingenol-3-mebutate for use as a medicament, including a medicament for the treatment of cancer or other condition involving neoplastic cells, or for the treatment of solar keratosis or actinic keratosis, or for the treatment of cancer wherein the cancer is skin cancer, melanoma, malignant melanoma, merkel cell carcinoma, squamous cell carcinoma, basal cell carcinoma or superficial basal carcinoma.

In another aspect, the invention provides a pharmaceutical composition comprising crystalline ingenol-3-mebutate or high-purity ingenol-3-mebutate, and one or more pharmaceutically acceptable carriers or vehicles. In certain embodiments, the pharmaceutical composition is suitable for topical administration.

In another aspect, the invention provides a pharmaceutical composition for topical application to a treatment area of a subject to deliver topically an effective amount of a crystalline ingenol-3-mebutate to treat a skin disorder in the treatment area, said pharmaceutical composition comprising an effective amount of a crystalline ingenol-3-mebutate and a pharmaceutically acceptable vehicle. In certain embodiments, the pharmaceutical composition is a gel, or a cream, or an ointment, or a suspension, or a lotion, or a salve, or a balm.

In another aspect, the invention provides a method of making a pharmaceutical composition comprising crystalline ingenol-3-mebutate, the method comprising combining crystalline ingenol-3-mebutate with a pharmaceutically acceptable carrier or vehicle.

In another aspect, the invention provides a process for preparing crystalline ingenol-3-mebutate, which comprises (a) dissolving an amount of ingenol-3-mebutate in a solvent or solvent mixture, (b) optionally adding water, (c) cooling the solution to a temperature at which about the full amount of ingenol-3-mebutate is no longer soluble in the solution, and (b) isolating by filtration any ingenol-3-mebutate crystals that are formed. In certain embodiments, the solvent is selected from C₁-C₆ linear or branched alkyl alcohols such as ethanol, and C₂-C₆ linear or branched alkyl nitriles such as acetonitrile.

In another aspect, the invention provides a method of treating cancer or other condition involving neoplastic cells, the method comprising administering an effective amount of a pharmaceutical composition according to the invention to a subject in need thereof.

In another aspect, the invention provides a method of treating actinic keratosis, the method comprising administering an effective amount of a pharmaceutical composition according to the invention to a subject in need thereof.

In another aspect, the invention provides a method of treating superficial basal cell carcinoma, the method comprising administering an effective amount of a pharmaceutical composition according to the invention to a subject in need thereof.

In another aspect, the invention provides a method of treating a wart selected from a group of warts consisting of common warts, genital warts and peri-anal warts, the method comprising administering an effective amount of a pharmaceutical composition according to the invention to a subject in need thereof.

In another aspect, the invention provides a method of treating photodamage, the method comprising administering an effective amount of a pharmaceutical composition according to the invention to a subject in need thereof.

In another aspect, the invention provides a topical method of treating a subject, who is diagnosed with actinic keratosis on the subject's face or scalp, with a gel formulated with about 0.015% ingenol-3-mebutate (ingenol-3-angelate), by weight, wherein localized skin responses caused by the gel are resolved within about two weeks following said topical method, said topical method comprises:
(a) squeezing a tube containing about 0.47 grams of the gel to dispense the gel onto a fingertip of the subject;
(b) applying the gel dispensed onto the fingertip to a treatment area on the subject's skin located on the subject's face or scalp, wherein the actinic keratosis is located, to topically treat the actinic keratosis in the treatment area, and wherein the treatment area is defined as one contiguous area of approximately 25 cm²;
(c) allowing the applied gel on the treatment area to dry for about 15 minutes;
(d) repeating said steps (a), (b) and (c) once daily for three consecutive days to treat the actinic keratosis on the subject's face or scalp;
(e) observing localized skin responses caused by the gel within about one day following the first day of said gel treatment;
(f) observing peak intensity of localized skin responses up to about one week following the 3rd day of said gel treatment; and
   (g) resolving localized skin responses within about two weeks following the 3rd day of said gel treatment.
In another aspect, the invention provides a tube containing about 0.47 grams of a gel formulated with about 0.05% ingenol-3-mebutate, by weight, for treating topically a subject, who is diagnosed with actinic keratosis in a treatment area on the body or extremities of the subject, wherein said subject
   (a) squeezes the tube containing about 0.47 grams of the gel to dispense the gel onto a fingertip of the subject;
   (b) applies the gel on the fingertip to the treatment area on the subject's skin on the subject's body or extremities to topically treat the actinic keratosis in the treatment area, wherein the treatment area is defined as one contiguous area of approximately 25 cm²;
   (c) allows the applied gel on the treatment area to dry for about 15 minutes; and
   (d) repeats said steps (a), (b) and (c) once daily for two consecutive days to treat the actinic keratosis in the treatment area on the subject's body or extremities.

In another aspect, the invention provides a topical method of treating a subject with a gel formulated with about 0.05% ingenol-3-mebutate, by weight, without observing systemic absorption of the ingenol mebutate, said method comprises:
(a) dispensing the gel contained within four individual tubes onto a treatment area on skin of the subject, wherein each said individual tube contains about 0.47 grams of the gel, and wherein the treatment area on skin of the subject is defined as one contiguous area of approximately 100 cm²; and
(b) repeating said step (a) once daily for two consecutive days without observing systemic absorption of ingenol-3-mebutate in the subject.

In another aspect, the invention provides a pharmaceutical gel composition comprising: (i) a solution of ingenol-3-mebutate prepared by dissolving crystalline ingenol-3-mebutate in a pharmaceutically acceptable solvent; and (ii) a gelling agent.

In another aspect, the invention provides a method of preparing an ingenol-3-mebutate pharmaceutical gel composition, the method comprising: (i) dissolving crystalline ingenol-3-mebutate in a pharmaceutically acceptable solvent to provide a solution of ingenol-3-mebutate; and (ii) combining the solution of ingenol-3-mebutate with a gelling agent, thereby preparing the ingenol-3-mebutate pharmaceutical gel composition.

In another aspect, the invention provides crystalline ingenol-3-mebutate characterized by an attenuated total reflectance fourier transform infrared (FTIR-ATR) spectrum substantially as shown in FIG. 1.

In another aspect, the invention provides crystalline ingenol-3-mebutate characterized by a structure obtained by single crystal X-Ray crystallography (XRC) substantially as shown in FIG. 2.

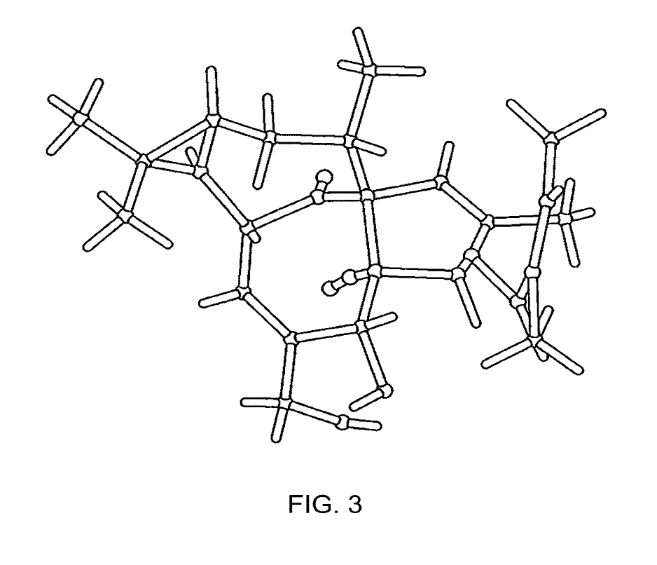

In another aspect, the invention provides crystalline ingenol-3-mebutate characterized by the crystal data and structure refinements substantially as shown in FIG. 3.

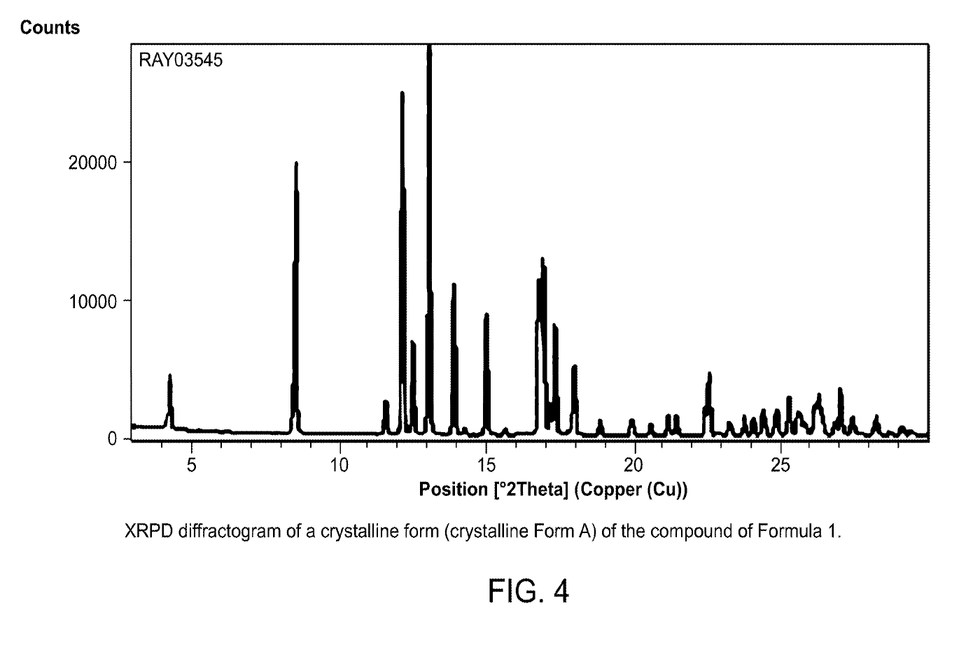

In another aspect, the invention provides crystalline ingenol-3-mebutate characterized by X-ray powder diffraction peaks substantially as shown in FIG. 4.

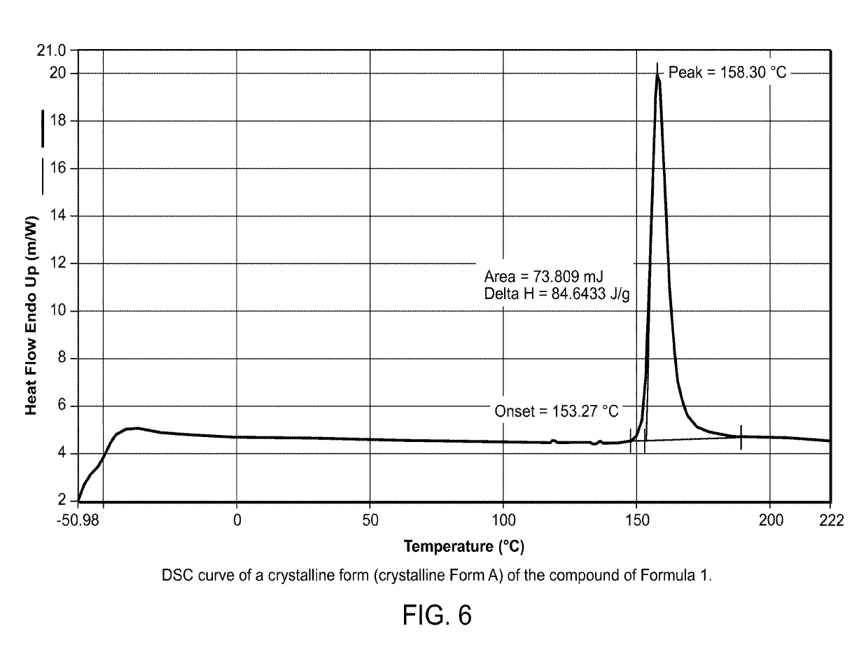

In another aspect, the invention provides crystalline ingenol-3-mebutate characterized by a differential scanning calorimetry (DSC) curve substantially as shown in FIG. 6.

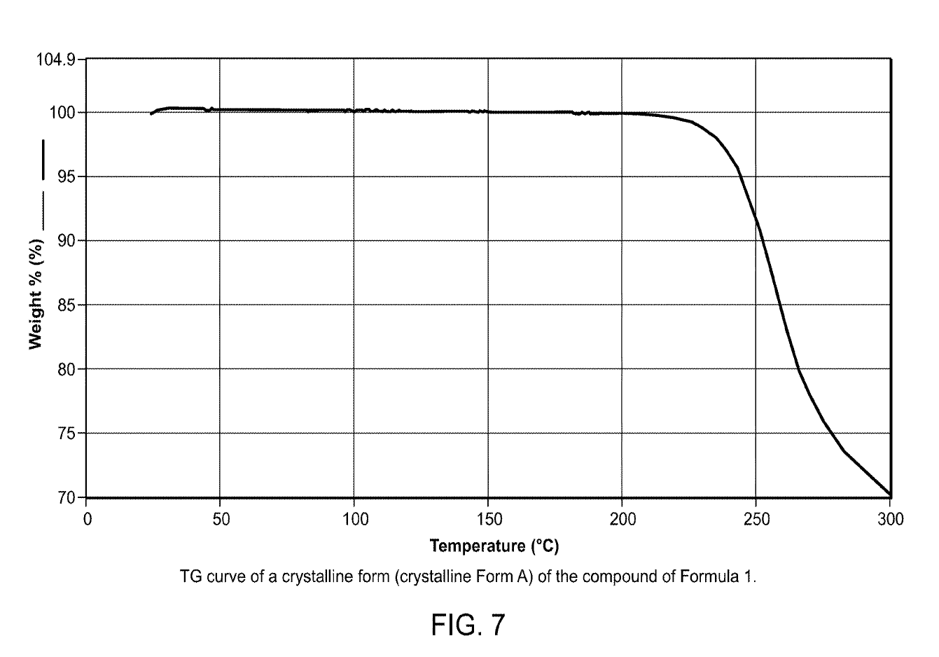

In another aspect, the invention provides crystalline ingenol-3-mebutate characterized by a thermogravimetric (TG) curve substantially as shown in FIG. 7.

In another aspect, the invention provides crystalline ingenol-3-mebutate characterized by Table 17 and/or Table 18.

In another aspect, the invention provides a topical pharmaceutical gel formulated with 0.05% ingenol mebutate (ingenol-3-angelate) by weight, wherein, when about 0.94 grams of the 0.05% ingenol mebutate pharmaceutical gel dispensed from two individual tubes is applied once a day on two consecutive days to a 100 cm² contiguous treatment area of skin of a patient, there is no detectable amount of the ingenol mebutate in the systemic circulation of the patient following the said topical application, wherein each individual tube is filled with about 0.47 grams of the 0.05% ingemol mebutate pharmaceutical gel.

In another aspect, the invention provides a topical pharmaceutical gel formulated with 0.015% ingenol mebutate (ingenol-3-angelate) by weight, wherein, when about 1.41 grams of the 0.015% ingenol mebutate pharmaceutical gel dispensed from three individual tubes is applied once a day on two consecutive days to a 100 cm² contiguous treatment area of skin of a patient, there is no detectable amount of the ingenol mebutate in the systemic circulation of the patient following the said topical application, wherein each individual tube is filled with about 0.47 grams of the 0.05% ingenol mebutate pharmaceutical gel.

In another aspect, the invention provides a method of topically treating actinic keratosis on a subject's face or scalp using a pharmaceutical gel formulation comprising about 0.015% ingenol-3-angelate, by weight, said topical method comprising:
(a) squeezing a tube containing about 0.47 grams of the pharmaceutical gel formulation to dispense at least some of the pharmaceutical gel formulation onto a fingertip of the subject;
(b) applying the dispensed pharmaceutical gel formulation to an actinic keratosis treatment area on the subject's face or scalp, wherein the actinic keratosis treatment area is one contiguous area of approximately 25 cm²;
(c) allowing the applied pharmaceutical gel formulation on the treatment area to dry for about 15 minutes;
(d) repeating said steps (a), (b) and (c) once daily for three consecutive days to treat actinic keratosis on the subject's face or scalp;
(e) observing localized skin responses, caused by applying the pharmaceutical gel formulation, within about one day following the first day of said gel treatment;
(f) observing peak intensity of said localized skin responses, up to about one week following the 3rd day of said gel treatment; and wherein said localized skin responses are resolved within about two weeks following the 3rd day of said topical treatment.

Additional aspects and embodiments of the invention are described herein.

### Pharmaceutical Formulations and Methods of Treatment

A further embodiment of the present invention encompasses pharmaceutical compositions comprising a crystalline form of the compound of Formula 1 and one or more pharmaceutically acceptable carriers or vehicles.

In one specific embodiment, the pharmaceutical compositions of the present invention are suitable for topical administration, especially ingenol mebutate gel pharmaceutical formulations.

A crystalline form of the compound of Formula I is useful in pharmaceutical formulations, including for example, wherein the ingenol mebutate is completely solubilized in an ingenol mebutate gel pharmaceutical formulation of the present invention or wherein the ingenol mebutate remains in crystalline form in a suspension.

Suspensions can be made from processed crystalline ingenol mebutate, such as micronized or nano-processed crystalline ingenol mebutate. A suspension may be used as it is in, for example, aerosols or processed into other pharmaceutical formulations such as creams, gels, ointments or other formulations useful for topical application.

In general, crystalline ingenol mebutate is dispersed in a vehicle. The vehicle can be water or another suitable vehicle, wherein the crystalline ingenol mebutate is maintained as a suspension and which has a viscosity suitable for delivery and for preventing the active substance from settling during storage.

Crystalline ingenol mebutate also may be dissolved in a solvent or solvent mixture, such as in a gel formulation.

Further additives may be stabilizers, emulsifiers, penetration enhancers, gelling agents and other components commonly used in dermal formulations, e.g., antioxidants, preservatives, pigments, skin soothing agents, skin healing agents and skin conditioning agents cf. CTFA Cosmetic Ingredients Handbook, 2^{nd} Ed., 1992. In one embodiment of the invention, the preservative is benzyl alcohol.

An embodiment of the present invention provides a gel formulation that comprises the use of crystalline ingenol mebutate as the API to formulate a pharmaceutical gel composition for treatment of skin conditions such as actinic keratosis or solar keratosis, or skin cancer as described herein. Concentration of the API in a pharmaceutical gel formulation is determined on the basis of the disease to be treated. For topical administration to treat skin conditions, such as actinic keratosis or solar keratosis, the API may be present in an amount of about 0.015% or about 0.05% by weight of the formulation. A further embodiment of the invention is the use of a crystalline form of the compound of Formula 1 as an API in a medicament.

Another embodiment of the present invention is the use of a crystalline form of the compound of Formula 1 as an API in a gel formulation for the treatment of actinic keratosis or solar keratosis, or seborrheic keratosis.

Another embodiment of the invention is the use of a crystalline form of the compound of Formula 1 as an API in a gel formulation for the treatment of skin cancer or other skin conditions involving neoplastic cells.

Another embodiment of the invention is the use of a crystalline form of the compound of Formula 1 as an API in a gel formulation for the treatment of basal cell carcinoma (BCC), nodular BCC, superficial basal cell carcinoma (sBCC), squamous cell carcinoma or squamous cell carcinoma in situ (SCCIS).

In embodiments of the invention, “skin cancer” is non-melanoma skin cancer, malignant melanoma, Merkel cell carcinoma, squamous cell carcinoma, squamous cell carcinoma, or basal cell carcinoma such as superficial basal cell carcinomas or nodular basal cell carcinoma.

The present invention will be exemplified by the following non-limiting examples.

### Examples

### Example 1: Preparation of Crystalline Ingenol Mebutate

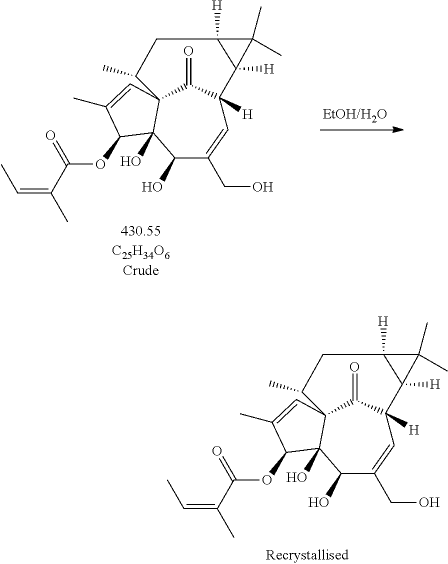

Four batches of crude Ingenol mebutate (about 22 g, 51.1 mmol) are combined in about a 500 ml vessel. Ethanol (about 154 ml) is charged to the vessel and the mixture is stirred at about 15 to 25° C. (the majority of crude ingenol mebutate was in solution after 5 minutes stirring). An oil bath is pre-heated to about 40° C. and the vessel is lowered into it (this effected full dissolution). Purified water (about 176 ml) is charged dropwise to the vessel until the mixture became permanently opaque. The mixture is stirred for about 5 to 6 minutes at about 30 to 35° C. (internal temperature), at this point the mixture is slightly opaque and a further charge of water is made (22 ml). The mixture is stirred for a further about 10 to 12 minutes at about 35 to 40° C. (internal temperature) then the heat source is removed and the opaque mixture is cooled to about 20 to 25° C. and stirred at this temperature for about 45 to 50 minutes. The reaction is further cooled to about 0 to 5° C. and stirred at this temperature for about 130 to 135 minutes. The suspension is filtered under vacuum and the collected solids are washed with half of the filtered recrystallisation liquors (used to rinse out the reaction vessel). The collected solids are further washed with purified water (2×110 ml) followed by heptanes (2×110 ml) and the filter cake is dried in vacuo for about 140 minutes. The collected solids are further dried in a vacuum oven at about 40 to 45° C. to afford Ingenol mebutate as a white solid (about 19.85 g, about 90% th, about 90% w/w). Purity by HPLC analysis: about 99.72 area %.

### Example 2: Preparation of Crystalline Ingenol Mebutate

Five lots of crude ingenol mebutate are combined into one pool of about 43.6 gram (containing about 28.07 gram of ingenol mebutate) by resuspension in acetone (about 233 mL) and is transferred to a round-bottled flask that is followed by evaporation to dryness on a rotary evaporator. The dried crude ingenol mebutate pool is added to about 38 mL of MeCN and is then slowly rotated for about 10 minutes on the rotary evaporator while it is heated using a waterbath (about 40° C.). This results in complete dissolution after which the temperature in the water-bath is decreased by about 5° C. at about every 25-30 minutes until the temperature is left at about 25° C. for about another 25 minutes. The flask is removed from the rotary evaporator and closed prior to placing it in the freezer at about −20° C. for about 11 days. Crystals had started to grow after about 5 days. The supernantant (about 15.5 mL) is removed by the use of a Pasteur pipette before the crystals of ingenol mebutate are collected on a PTFE filter membrane using vacuum filtration. The crystallisation flask is rinsed using pre-cooled MeCN (24 mL) and the crystals are spread to cover the filter evenly before additional drop-wise washing using pre-cooled MeCN (2×24 mL) is conducted. Upon completion, the crystals are partially dried by vacuum for about 15 minutes followed by a flow of nitrogen for about 1½ hr. This results in about 10.9 grams of white to off-white or pale yellow ingenol mebutate crystals (about 39% yield based on ingenol mebutate).

### Example 3: XRC Single Crystal Structure of Crystalline Ingenol Mebutate

A representative colourless rod-shaped crystal (about 0.3 mm×about 0.05 mm×about 0.05 mm) obtained using the method of Example 1 is surveyed and a data set is collected on a NoniusKappaCCD area detector diffractometer. Other experimental details: Diffractometer: Nonius KappaCCD area detector (φ scans and ω scans to fill asymmetric unit). Cell determination: DirAx (Duisenberg, A J. M. (1992). J Appl. Cryst. 25, 92-96.) Data collection: Collect (Collect: Data collection software, R Hooft, Nonius B. V., 1998). Data reduction and cell refinement: Denzo (Z. Otwinowski & W. Minor, Methods in Enzymology (1997) vol. 276: Macromolecular Crystallography part A, pp. 307-326; C. W. Carter, Jr. & R. M. Sweet, Eds., Academic Press). Absorption correction: Sheldrick, G. M. SADABS—Bruker Nonius area detector scaling and absorption correction—V2.10 Structure solution: SHELXS97 (G. M. Sheldrick, Acta Cryst. (1990) A46 467-473). Structure refinement: SHELXS97 (G. M. Sheldrick (1997), University of Gottingen, Germany). Graphics: Cameron—A Molecular Graphics Package. (D. M. Watkin, L. Pearce and C. K Prout, Chemical Crystallography Laboratory, University of Oxford, 1993).

Special details: All hydrogen atoms are placed in idealized positions and realigned using a riding model. There is conformational disorder in the macrocyclic ring.

Details of the crystal are provided by Table 1 above. Selected atomic coordinates and isotropic thermal parameters determined from the data are provided in Table 2. Bond lengths and Bond angles are provided in Table 3. Other crystal data and structure refinement details are provided in Table 4.

TABLE 2
Atomic coordinates [×10⁴], equivalent isotropic displacement
parameters [Å² × 10³] and site occupancy factors. U_{eq}
is defined as one third of the trace of the orthogonalized U^{ij} tensor.

| Atom | x | y | z | U_{eq} | S.o.f. |
|---|---|---|---|---|---|
| C1 | 3825(5) | 3707(5) | 428(1) | 38(1) | 1 |
| C2 | 5900(5) | 3903(5) | 425(1) | 37(1) | 1 |
| C3 | 7212(5) | 2727(5) | 453(1) | 33(1) | 1 |
| C4 | 9278(5) | 3151(6) | 417(1) | 50(1) | 1 |
| C5 | 6793(5) | 850(5) | 497(1) | 35(1) | 1 |
| C6 | 4864(6) | −1236(4) | 772(1) | 33(1) | 1 |
| C7 | 2887(6) | −1430(5) | 656(1) | 46(1) | 1 |
| C8 | 2444(8) | −1555(5) | 302(1) | 62(1) | 1 |
| C9 | 1694(6) | −1429(5) | 902(1) | 49(1) | 1 |
| C10 | 2614(5) | −1194(4) | 1225(1) | 32(1) | 1 |
| C11 | 4774(5) | −1556(4) | 1144(1) | 27(1) | 1 |
| C12A | 213(9) | 1111(9) | 1274(1) | 35(1) | 0.539(2) |
| C13A | 2274(10) | 698(9) | 1325(1) | 35(1) | 0.539(2) |
| C14A | 2828(9) | 1265(8) | 1668(1) | 35(1) | 0.539(2) |
| C15A | 1838(10) | 382(9) | 1957(1) | 35(1) | 0.539(2) |
| C16A | 2919(10) | −191(9) | 2256(2) | 35(1) | 0.539(2) |
| C17A | 2113(10) | −1546(9) | 2025(2) | 35(1) | 0.539(2) |
| C20A | 1909(10) | −75(9) | 2578(1) | 35(1) | 0.539(2) |
| C21A | 4992(9) | 65(9) | 2283(1) | 35(1) | 0.539(2) |
| C18A | 3229(5) | −2388(5) | 1771(1) | 35(1) | 0.539(2) |
| C12B | 1705(11) | 2107(10) | 1216(2) | 35(1) | 0.461(2) |
| C13B | 2166(12) | 586(9) | 1441(2) | 35(1) | 0.461(2) |
| C14B | 549(11) | 552(11) | 1702(2) | 35(1) | 0.461(2) |
| C15B | 606(12) | −493(10) | 2018(2) | 35(1) | 0.461(2) |
| C16B | 2124(12) | −533(11) | 2279(2) | 35(1) | 0.461(2) |
| C17B | 1868(12) | −2096(11) | 2067(2) | 35(1) | 0.461(2) |
| C20B | 1363(12) | −759(11) | 2623(2) | 35(1) | 0.461(2) |
| C21B | 3804(11) | 657(10) | 2260(2) | 35(1) | 0.461(2) |
| C18B | 3229(5) | −2388(5) | 1771(1) | 35(1) | 0.461(2) |
| C19 | 1975(5) | −2445(4) | 1474(1) | 30(1) | 1 |
| C22 | 4360(5) | −3988(4) | 1775(1) | 30(1) | 1 |
| C23 | 5375(5) | −4415(4) | 1518(1) | 26(1) | 1 |
| C24 | 5371(5) | −3501(4) | 1188(1) | 28(1) | 1 |
| C25 | 6607(5) | −5983(4) | 1526(1) | 33(1) | 1 |
| O1 | 7605(5) | −262(4) | 356(1) | 66(1) | 1 |
| O2 | 5432(4) | 550(3) | 717(1) | 30(1) | 1 |
| O3 | 6091(3) | −592(3) | 1322(1) | 28(1) | 1 |
| O4 | 587(3) | −3356(4) | 1454(1) | 41(1) | 1 |
| O5 | 7194(3) | −3705(3) | 1045(1) | 31(1) | 1 |
| O6 | 6344(4) | −7081(3) | 1249(1) | 38(1) | 1 |

TABLE 3
Bond lengths [A]

| | |
|---|---|
| C1—C2 | 1.488(5) |
| C2—C3 | 1.311(5) |
| C3—C5 | 1.497(5) |
| C3—C4 | 1.516(5) |
| C5—O1 | 1.191(5) |
| C5—O2 | 1.350(4) |
| C6—O2 | 1.461(4) |
| C6—C7 | 1.496(6) |
| C6—C11 | 1.560(4) |
| C7—C9 | 1.327(6) |
| C7—C8 | 1.502(5) |
| C9—C10 | 1.502(5) |
| C10—C19 | 1.487(5) |
| C10—C13A | 1.543(7) |
| C10—C11 | 1.602(5) |
| C10—C13B | 1.675(8) |
| C11—O3 | 1.409(4) |
| C11—C24 | 1.578(5) |
| C12A—C13A | 1.519(10) |
| C13A—C14A | 1.539(8) |
| C14A—C15A | 1.547(9) |
| C15A—C16A | 1.524(9) |
| C15A—C17A | 1.534(9) |
| C16A—C21A | 1.496(10) |
| C16A—C20A | 1.519(9) |
| C16A—C17A | 1.532(9) |
| C17A—C18A | 1.472(7) |
| C18A—C22 | 1.480(5) |
| C18A—C19 | 1.519(4) |
| C12B—C13B | 1.538(10) |
| C13B—C14B | 1.580(10) |
| C14B—C15B | 1.539(10) |
| C15B—C16B | 1.530(11) |
| C15B—C17B | 1.548(11) |
| C16B—C17B | 1.509(11) |
| C16B—C21B | 1.514(11) |
| C16B—C20B | 1.533(10) |
| C19—O4 | 1.219(4) |
| C22—C23 | 1.329(4) |
| C23—C25 | 1.500(5) |
| C23—C24 | 1.538(4) |
| C24—O5 | 1.436(4) |
| C25—O6 | 1.440(4) |

Bond angles [°]

| | |
|---|---|
| C3—C2—C1 | 129.6(4) |
| C2—C3—C5 | 123.0(3) |
| C2—C3—C4 | 122.2(4) |
| C5—C3—C4 | 114.6(3) |
| O1—C5—O2 | 123.7(3) |
| O1—C5—C3 | 123.3(3) |
| O2—C5—C3 | 113.1(3) |
| O2—C6—C7 | 107.8(3) |
| O2—C6—C11 | 108.5(2) |
| C7—C6—C11 | 105.2(3) |
| C9—C7—C6 | 110.9(3) |
| C9—C7—C8 | 127.9(4) |
| C6—C7—C8 | 121.2(4) |
| C7—C9—C10 | 113.9(3) |
| C19—C10—C9 | 113.9(3) |
| C19—C10—C13A | 112.8(3) |
| C9—C10—C13A | 106.6(3) |
| C19—C10—C11 | 109.0(3) |
| C9—C10—C11 | 102.1(3) |
| C13A—C10—C11 | 112.0(4) |
| C19—C10—C13B | 96.3(3) |
| C9—C10—C13B | 119.5(4) |
| C11—C10—C13B | 116.1(4) |
| O3—C11—C6 | 113.8(3) |
| O3—C11—C24 | 105.5(2) |
| C6—C11—C24 | 104.8(2) |
| O3—C11—C10 | 115.9(3) |
| C6—C11—C10 | 102.7(3) |
| C24—C11—C10 | 113.8(3) |
| C12A—C13A—C14A | 108.4(5) |
| C12A—C13A—C10 | 108.4(5) |
| C14A—C13A—C10 | 118.6(5) |
| C13A—C14A—C15A | 118.0(5) |
| C16A—C15A—C17A | 60.1(4) |
| C16A—C15A—C14A | 121.7(6) |
| C17A—C15A—C14A | 121.0(6) |
| C21A—C16A—C20A | 113.2(5) |
| C21A—C16A—C15A | 121.5(6) |
| C20A—C16A—C15A | 117.1(6) |
| C21A—C16A—C17A | 120.6(6) |
| C20A—C16A—C17A | 114.2(6) |
| C15A—C16A—C17A | 60.3(4) |
| C18A—C17A—C16A | 123.2(6) |
| C18A—C17A—C15A | 111.8(5) |
| C16A—C17A—C15A | 59.6(4) |
| C17A—C18A—C22 | 131.2(4) |
| C17A—C18A—C19 | 105.7(4) |
| C22—C18A—C19 | 107.8(3) |
| C12B—C13B—C14B | 105.7(6) |
| C12B—C13B—C10 | 110.5(5) |
| C14B—C13B—C10 | 119.3(6) |
| C15B—C14B—C13B | 124.7(7) |
| C16B—C15B—C14B | 129.0(7) |
| C16B—C15B—C17B | 58.7(5) |
| C14B—C15B—C17B | 123.3(6) |
| C17B—C16B—C21B | 123.6(7) |
| C17B—C16B—C15B | 61.2(5) |
| C21B—C16B—C15B | 120.6(6) |
| C17B—C16B—C20B | 114.0(7) |
| C21B—C16B—C20B | 113.6(6) |
| C15B—C16B—C20B | 114.1(7) |
| C16B—C17B—C15B | 60.0(5) |
| O4—C19—C10 | 125.3(3) |
| O4—C19—C18A | 123.5(3) |
| C10—C19—C18A | 111.2(3) |
| C23—C22—C18A | 119.8(3) |
| C22—C23—C25 | 120.2(3) |
| C22—C23—C24 | 126.5(3) |
| C25—C23—C24 | 113.2(3) |
| O5—C24—C23 | 108.2(3) |
| O5—C24—C11 | 107.6(3) |
| C23—C24—C11 | 123.0(3) |
| O6—C25—C23 | 112.7(3) |
| C5—O2—C6 | 117.9(3) |

### Example 4: Additional Characterization of Crystalline Ingenol Mebutate

### Materials

Ingenol mebutate Batch X08014RP

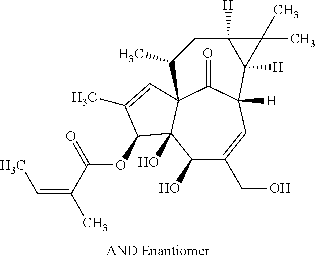 [SMILES: C/C=C(/C)C(=O)O[C@H]1C(C)=C[C@]23C(=O)[C@@H](C=C(CO)[C@@H](O)[C@]12O)[C@H]1[C@@H](C[C@H]3C)C1(C)C]

### Equipment

### X-Ray Powder Diffraction

The X-ray powder diffractograms are recorded on a PANalytical diffractometer with monochromatic Cu—K_{α} radiation. Samples are measured in transmission. The generator settings are about 45 kV and about 40 mA. Soller slits of about 0.02 rad are used both in the incident and in the diffracted beam path. In the incident beam path, an anti scatter slit of about 1°, a divergence slit of about ½° and a mask of 4 mm are used. In the diffracted beam path, an anti scatter slit of about 2.0 mm is used. The measuring conditions are: about 20=about 5-25°, step size about 0.0066°, time per step about 400.350 seconds. The samples are measured on a zero background high-throughput stage sample holder and are oscillated along the X axis in the range of about 1 mm. A PIXcel[1] detector is used.

### Proton (¹H)-NMR

Solution ¹H NMR experiments are performed to identify and quantify the amount of solvent present on the screening sample. ¹H NMR experiments are performed on a Bruker Avance-600 MHz NMR spectrometer. Deuterated methanol is used as solvent. The acquisition time is about 2.7 sec. About 64 scans are acquired before Fourier transformation.

### Raman Spectroscopy

The FT-Raman spectra are recorded using a Bruker RFS 100/S FT-Raman spectrometer equipped with about a 1064 nm Adlas DPY 421 Nd:YAG laser with a maximum power of about 1550 mW and a liquid nitrogen cooled Ge detector. For each sample, 128 scans are collected using a focussed beam (laserspot about 100 μm), a laserpower of about 150 mW and a resolution of about 2 cm⁻¹.

### Differential Scanning Calorimetry (DSC)

The DSC curves are recorded using a Perkin Elmer DSC 8500, closed aluminium crucibles with a volume of about 40 μL in combination with a nitrogen flow of about 20 mL/min. The heating rate that is used is about 20° C./min.

### Thermogravimetry (TG)

The TG curve is recorded to determine the amount of solvent present on the screening samples. The curves are measured using a Perkin Elmer Pyris 1 TGA instrument. Closed pierced aluminium pans are used in combination with a nitrogen flow of about 50 mL/min. The heating rate is about 10° C./min.

### Microscopy Analysis

The microscopy pictures are recorded using an Olympus BX51 that is equipped with a ×10 objective, circular Polaroid filters and JVC colour digital video camera.

### HPLC Purity Analysis

Purity of ingenol mebutate can be determined using the following HPLC method:

Column: Symmetry C18-5 μm (Waters)

Column length: 150×3.9 mm

Column temperature: 30° C.

Guard column: Symmetry C18-5 μm (Waters)

Guard column length: 20×3.9 mm

Mobile phase:

0.02% v/v TFA in water (A);

0.02% v/v TFA in Acetonitrile (B)

Gradient (min/% B) 0/50, 2/50, 5/60, 12/80, 16/80, 16.5/50, 20/50, 25/50

Flow rate: 1.0 mL/min

Sampler temperature: 5° C.±3° C.

UV wavelength: 230 nm

Injection volume: 10 mL

Run time: 20 min

The retention time of the main ingenol mebutate peak using this method is found to be approximately 9 min.

### Methods

Crystallisation experiments are carried out in about 4 mL vials. The solutions are stirred at approximately 550 rpm. Three different crystallisation methods are used: fast cooling, evaporation and anti-solvent crystallisations.

### Fast Cooling Crystallisation

A rapid induced cooling is achieved by submerging the solutions in a liquid nitrogen bath. The crystals are filtered, dried at room temperature in vacuum. As for solutions where crystallisation is not initially observed, they are placed in the freezer. The experiments are performed using 3 different solvents. Only two yielded crystalline material.

### Evaporation of Solvent

Complete or near complete evaporation of the solvents are performed at room temperature without the use of nitrogen. After a considerable (sufficiently) amount of crystals is observed, the crystals are isolated and dried at room temperature in vacuum. The experiments are performed using a total of about 32 solvents and combinations of these solvents. Several solvents and combinations of these solvents produced crystalline material (see, e.g., Table 5, below).

### Anti-Solvent Crystallisation

In this study, the saturated solutions at various respective volumes are added to an excess volume of anti-solvent. Seven anti-solvent experiments are performed using five different solvents and two different anti-solvents. All the experiments do produce crystalline material after stirring for a certain time. Due to the substantial amount of time elapsed before crystallization occurs in water, this is not a normal anti-solvent crystallization. The crystals are isolated and dried to constant weight at room temperature in vacuum.

The performed crystallization experiments are tabulated in Table 5.

### Milling

Crystalline ingenol mebutate is subjected to stress milling to investigate its physico-chemical stability. In the study, crystalline ingenol mebutate is manually ground using a mortar and pestle for durations of about 2, about 5 and about 10 mins.

### Amorphization

To produce amorphous material, ingenol mebutate is dissolved in dichloromethane and the solution is allowed to evaporate at room temperature. White residual compound is found after one day.

TABLE 5
Summary of crystallisation experiments for ingenol mebutate.

| Sample | Solvent | Crystallisation method | Crystals |
|---|---|---|---|
| COS1001A | Acetone | Evaporation of solvent | Yes |
| COS1001B | Ethanol (EtOH) | Evaporation of solvent | Yes |
| COS1001C | Methanol (MeOH) | Evaporation of solvent | |
| COS1001D | Ethylacetate | Evaporation of solvent | |
| COS1001E | Toluene | Evaporation of solvent | Yes |
| COS1001F | Dioxane (Diox) | Evaporation of solvent | |
| COS1001G | Tetrahydrofuran | Evaporation of solvent | |
| | (THF) | | |
| COS1001H | Dichloromethane | Evaporation of solvent | |
| COS1001J | 1-propanol | Evaporation of solvent | |
| COS1001K | 2-propanol (2PrOH) | Evaporation of solvent | Yes |
| COS1001L | Acetonitrile (ACN) | Evaporation of solvent | Yes |
| COS1001M | Diethylether | Evaporation of solvent | |
| COS100N | 1:1 MeOH:Water | Evaporation of solvent | |
| COS100P | 1:1 EtOH:Water | Evaporation of solvent | Yes |
| COS100Q | 1:1 Diox:heptane | Evaporation of solvent | |
| COS100R | heptane | Evaporation of solvent | Yes |
| COS100T | c-hexane + 2 drops | Evaporation of solvent | Yes |
| | acetone | | |
| COS100U | pentane + 2 drops | Evaporation of solvent | |
| | acetone | | |
| COS100V | n-hexane + 2 drops | Evaporation of solvent | |
| | acetone | | |
| COS1001W | 2-butanone | Evaporation of solvent | |
| COS1001X | Benzyl alcohol | Evaporation of solvent | |
| COS1001Y | MTBE | Evaporation of solvent | Yes |
| COS1003A | c-hexane | Evaporation of solvent | Yes |
| COS1004E | DMSO | Evaporation of solvent | Yes |
| COS1004F | Monoglyme | Evaporation of solvent | Yes |
| COS1004A | Toluene | Fast cooling | Yes |
| COS1004B | ACN | Fast cooling | Yes |
| COS1004C | 2PrOH | Fast cooling | |
| COS1003B | ACN/water | Anti solvent crystallisation | Yes |
| COS1003C | Toluene/heptane | Anti solvent crystallisation | Yes |
| COS1003D | Dioxane/heptane | Anti solvent crystallisation | Yes |
| COS1003E | 2PrOH/water | Anti solvent crystallisation | Yes |
| COS1003E1 | 2PrOH/heptane | Anti solvent crystallisation | Yes |
| COS1003G | Acetone/heptane | Anti solvent crystallisation | Yes |
| COS1004D | Dioxane/heptane + | Anti solvent crystallisation | Yes |
| | DMSO | | |
| COS1006A | Xylene + 5 drops | Evaporation of solvent | Yes |
| | acetone | | |
| COS1006B | i-octane + 5 drops | Evaporation of solvent | Yes |
| | acetone | | |
| COS1006C | Diglyme | Evaporation of solvent | |
| COS1006D | Cumene | Evaporation of solvent | |
| COS1006E | Anisole | Evaporation of solvent | |
| COS1006F | Pyridine | Evaporation of solvent | |
| COS1006G | Sulfalone | Evaporation of solvent | |

### Screening Results

To investigate the polymorphism of crystalline ingenol mebutate, a variety of crystallization experiments were performed using different solvents (as described herein) and crystallisation methods.

The samples were analyzed by X-ray powder diffraction (XRPD) Raman and differential scanning calorimetry (DSC) to determine the polymorphic form and by ¹H nuclear magnetic resonance spectroscopy (1H NMR) and differential thermal analysis (TG) to determine the existence of solvates.

In the screening experiments, one main crystalline form of ingenol mebutate, named “form A”, was detected. Furthermore, two solvates, named “form X” and “form Y”, were found. Finally, ingenol mebutate can be present in amorphous form.

From the crystallization experiments (see Table 5), form A was shown to crystallize from a selected number of solvents (ethanol, methyl t-butyl ether (MTBE), toluene, heptane, etc). As for the 2 solvates, their crystallisation depended on which solvent was used, e.g., forms X and Y crystallized from cyclohexane and DMSO, respectively. Crystallization from dichloromethane resulted in solid amorphous material. Temperature tampering of the amorphous material (e.g. heating to about 80° C. and cooling to about 20° C.) did not yield any crystalline material.

A solvent-mediated conversion experiment of forms A, X and Y was performed to determine the most stable form at room temperature. The result indicated form A to be the most stable form at room temperature.

Crystalline ingenol mebutate was subjected to stress milling (manually using a mortar and pestle) for durations of about 2, about 5 and about 10 mins. Form A remained unaltered during the milling process.

### Characterisation of the Polymorphic Forms

### Form A

Form A was characterised by FTIR-ATR (FIG. 1), single crystal X-ray crystallography (FIG. 2), crystal data and structure refinements (FIG. 3 and Table 4), XRPD (FIG. 4), Raman (FIG. 5), DSC (FIG. 6) and TG (FIG. 7). The average melting point of form A, determined by DSC, was about 153.5±5° C., and the average enthalpy was about 85±8 mJ/mg. In general, the variation in the melting point and average enthalpy was attributed to differences in particle size of the samples.

### Amorphous Form

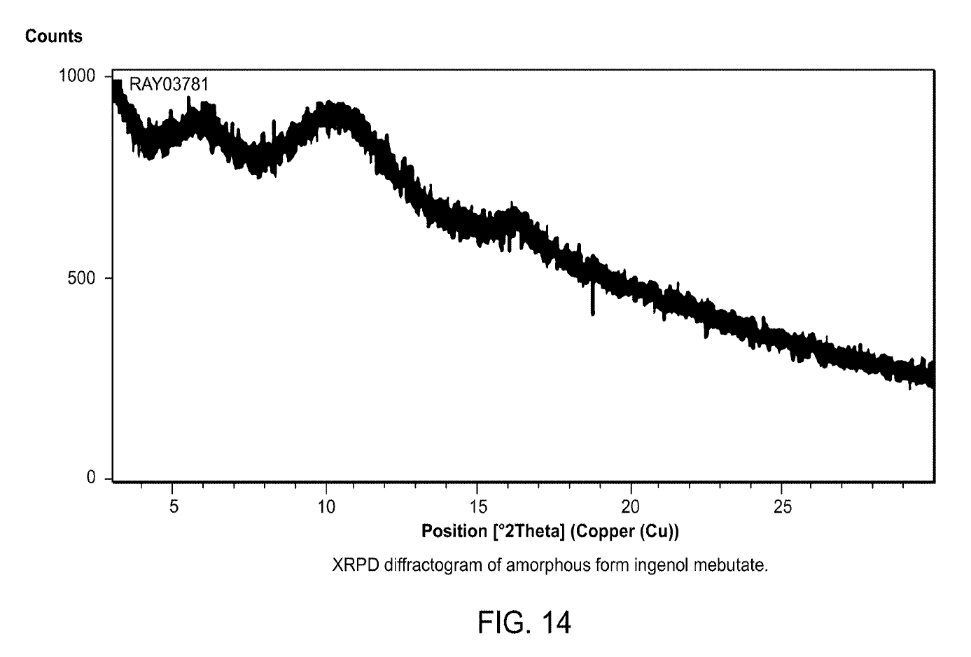

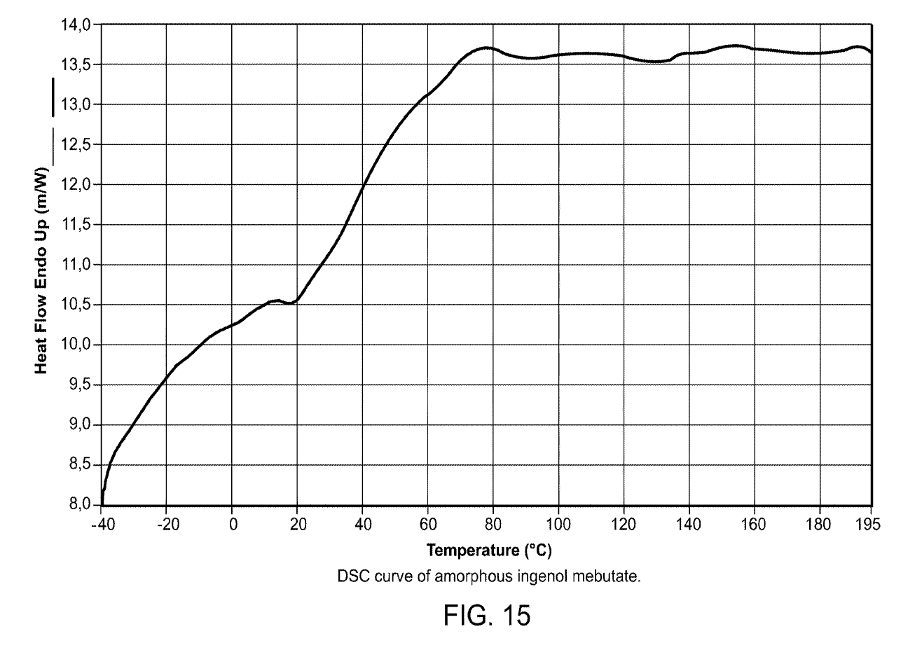

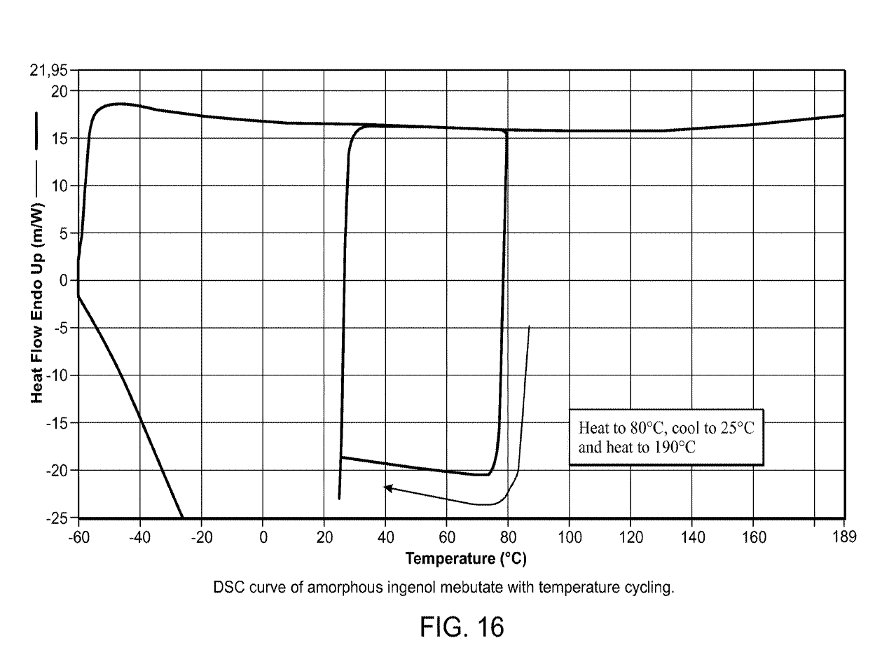

Amorphous ingenol mebutate was characterized by XRPD (see FIG. 14) and DSC (FIG. 15). A temperature tampering process is shown in FIG. 16.

### Slurry Experiment

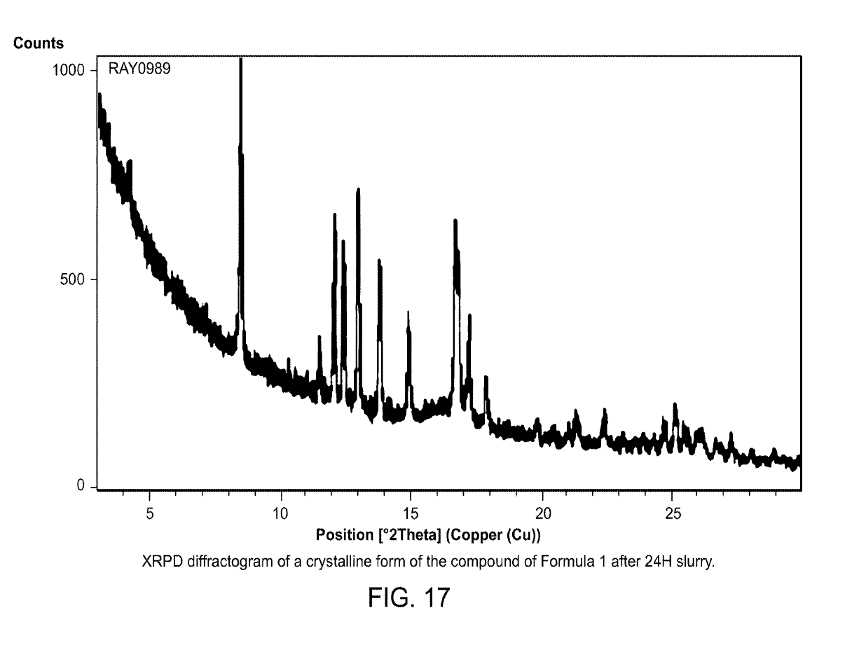

The fact that crystalline form A was the most stable form at room temperature was confirmed with the slurry experiment. For the experiment, a slurry mixture of forms A, X and Y in cyclohexane was stirred for about 48 hours at room temperature. After about 24 hours, the complete sample was transformed into form A (see FIG. 17).

### Milling Experiment

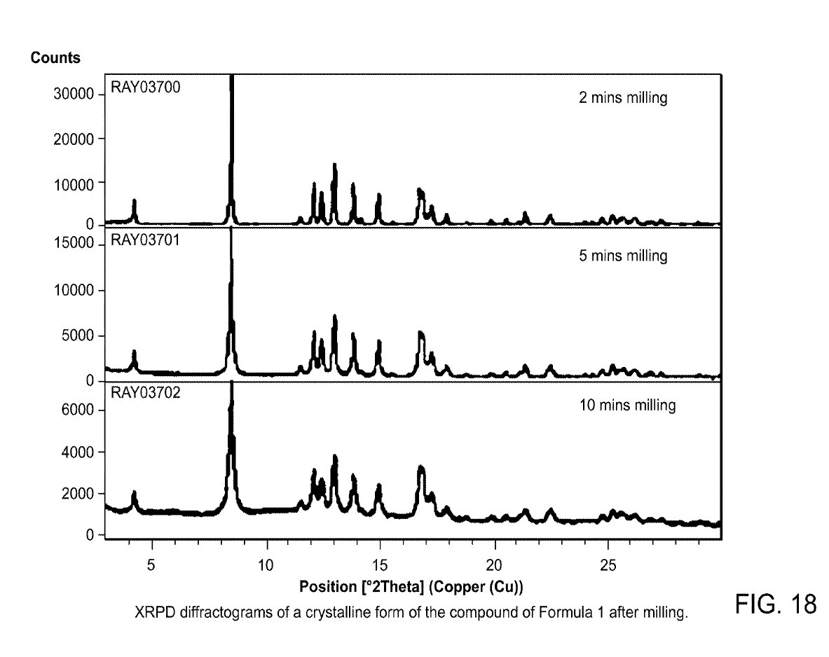

Crystalline ingenol mebutate was subjected to stress milling (manually using a mortar and pestle) for durations of about 2, about 5 and about 10 mins. Form A remained unaltered during the milling process (see FIG. 18).

### Conclusions

Main form:

A polymorphic crystalline form of the compound of Formula 1 was characterized and identified as form A″ The stability of form A at room temperature was confirmed by the slurry experiments at room temperature. These experiments demonstrated that solvate forms X and Y, in a slurry suspension of form A and solvate forms X and Y in c-hexane, uniquely converted to form A after about 24 hours.

### Solvates:

In addition to the main polymorphic form A, two solvates were detected. The minor solvate form called form X was characterized by only 2 single small peaks at about 3.9° and about 5.5° 2θ in a XRPD diffractogram. The other minor solvate form called form Y was characterised by peaks at about 5.1°, about 6.3° and about 10.7° 2θ in a XRPD diffractogram.

Solvate forms X and Y converted into form A upon heating. The solvent mediated conversion experiment of forms A, X and Y indicated that form A was the most stable polymorphic form at room temperature.

### Example 5: Pharmaceutical Formulations

Examples of pharmaceutical formulations of the present invention include topical pharmaceutical gels formulated with, e.g., (i) 0.015% or 0.05% by weight of the gel formulation of a crystalline form of the compound of Formula 1, (ii) isopropyl alcohol, (iii) hydroxyethylcellulose, (iv) citric acid monohydrate, (v) sodium citrate dihydrate, (vi) benzyl alcohol and (vii) purified water.

Exemplary bulk gel formulations contemplated by the present invention:

0.015% or 0.05% by weight of the gel formulation of a crystalline form of the compound of Formula 1,

Ingenol mebutate

isopropyl alcohol

hydroxyethylcellulose

citric acid monohydrate

sodium citrate dihydrate

benzyl alcohol

purified water

In certain specific embodiments, the following formulations are used:

A. Ingenol mebutate (0.015% w/w) is formulated with the following excipients:

| Excipient | Concentration (%) | Function |
|---|---|---|
| Benzyl Alcohol NF | 0.90 | Solubiliser/Preservative |
| Isopropyl Alcohol USP | 30.0 | Solubiliser/volatile phase |
| Citric Acid USP | 0.56 | Buffering Agent |
| Sodium Citrate | 0.14 | Buffering Agent |
| Dihydrate USP | | |
| Hydroxyethyl Cellulose | 1.5 | Viscosity Modifier |
| NF | | |
| Purified Water USP | q.s. ad 100 | Vehicle |

B. Ingenol mebutate (0.05% w/w) is formulated with the following excipients:

| Excipient | Concentration (%) | Function |
|---|---|---|
| Benzyl Alcohol NF | 0.90 | Solubiliser/Preservative |
| Isopropyl Alcohol USP | 30.0 | Solubiliser/volatile phase |
| Citric Acid USP | 0.56 | Buffering Agent |
| Sodium Citrate | 0.14 | Buffering Agent |
| Dihydrate USP | | |
| Hydroxyethyl Cellulose | 1.5 | Viscosity Modifier |
| NF | | |
| Purified Water USP | q.s. ad 100 | Vehicle |

The container closure system is a unit-dose system consisting of a laminate tube with a polyethylene screw cap. In one embodiment, the tube may be filled with, e.g., about 0.47 grams to about 0.51 grams, or about 0.47 grams, of a topical pharmaceutical composition formulated with ingenol mebutate, as contemplated by the present invention, e.g., such as the formulations A or B above.

In the treatment of, for example, actinic keratosis on the face and/or scalp of a subject, a 0.015% ingenol mebutate topical gel of the present invention may be applied on the face and scalp to the affected skin area (treatment area) once a day for 3 consecutive days.

In the treatment of, for example, actinic keratosis on the trunk and/or extremities of a subject, a 0.05% ingenol mebutate topical gel of the present invention may be applied on the trunk and extremities to the affected skin area (treatment area) once a day for 2 consecutive days.

A treatment area can be defined, for example, as one contiguous area of approximately 25 cm² (e.g., 5 cm×5 cm). The gel from for example a unit dose tube or package containing approximately 0.47 g of the gel, can be squeezed onto the fingertip and spread evenly over the entire treatment area, allowing the gel to dry for about 15 minutes. Preferably, one unit dose tube (tube with screw cap or individual packets) may be used for one treatment area. Immediately, following application of a gel to the treatment area, subjects should wash their hands.

Under maximum use conditions, e.g., when an about 100 cm² contiguous treatment area is topically treated with 4 unit doses of 0.05% ingenol mebutate gel once daily for 2 consecutive days, it is believed that there is little to no systemic absorption of the ingenol mebutate. Thus, it is contemplated by the present invention that up to at least about 2 unit dose tubes, each filled with 0.05% ingenol mebutate gel in an amount of about 0.47 grams, or about 6 unit dose tubes, each filled with 0.015% ingenol mebutate gel in an amount of about 0.47 grams, may be applied to a treatment area once daily for 2 consecutive days, that totals a maximum treatment area affected with actinic keratosis of about 100 cm², without causing treatment limiting systemic absorption of ingenol mebutate.

### Example 6: Formulation Release Rates

### I. In-Vitro Release Study: Comparison of Cream and Gel Prototype

Absorption of drugs into or through the skin depends upon a number of factors including composition of the vehicle, type and condition of the skin and external factors (temperature, humidity, and occlusion). However, the factor with perhaps the greatest influence on the rate or extent of percutaneous absorption is the thermodynamic activity of the drug, which is influenced by its physicochemical properties such as stability, solubility, molecular size, log P and ionisation state.

Previously synthetic membranes have been investigated as a readily available and easy-to-use tool to study the in vitro release profiles of drugs from topical formulations in order to ascertain batch to batch uniformity of dermatological products.

Higuchi's equation of mass transport is widely accepted as an appropriate model drug for permeation

across membranes (Equation 1),

$$J = \frac{α DA}{γ L} \qquad ( 1 )$$

where J is the flux of the molecule, a is the thermodynamic activity of the drug in the donor solution, D is the diffusion coefficient of the drug, A is the effective cross section area of the membrane, and y is the effective activity coefficient of the drug in the membrane. As can be seen the flux of the molecule across a membrane is directly proportional to the thermodynamic activity of the drug in a formulation. Where a drug is formulated at its maximum solubility in the formulation TA=1 and as such the method described is an ideal way of comparing drug release between formulations and ultimately assessing the ability of the formulation to present the drug at the skin interface ready for partitioning into and diffusion across the stratum corneum.

A comparison of the in-vitro release of the drug substance from the hydroalcoholic gel and macrocetyl ether cream formulation across a synthetic membrane was investigated using Franz diffusion cells under non-occluded conditions. Both formulations contained 0.1% w/w ingenol mebutate. The ointment was rejected at the time of performing the release study due to observed ingenol mebutate precipitation at the 6 months time point. The greatest total release of ingenol mebutate for the duration of the in-vitro release study was observed for the gel formulation. After an initial lag time a steady state of release of ingenol mebutate was achieved for the gel from 3 hours up to approximately 10 hours, after which, a slower rate of release was observed, which is attributed to the evaporation of the volatile phase of the gel whereby the formulation is drying out on the membrane.

The release of ingenol mebutate from the cream formulation was much less than that observed from the gel formulation in the total study period. The lag time observed for the cream formulation was 7 hours which is greater than the lag time observed for the gel formulation (3 hours). Note that the presence of the lag time can be considered to arise because of two reasons; (i) the amount of PEP005 in the receiver fluid at these time points was below the LOQ/LOD for the method and/or (ii) it is the time taken for the ingenol mebutate to partition from the formulation into the membrane.

The release data for ingenol mebutate from the cream and gel formulations are detailed in Table 6 below.

TABLE 6

| | | Release | Total Amount Released |
|---|---|---|---|
| | Time Interval | Rate | Mean ± SEM n = 3 |
| Formulation | (hours) | μg/cm²h⁻¹ | (μg/cm²) |
| 0.1% w/w ingenol | 2-10 | 9.17 | 133.38 ± 5.44 |
| mebutate gel | | | (T = 26 hours) |
| 0.1% w/w ingenol | 7-24 | 0.53 | 8.92 ± 0.77 |
| mebutate cream | | | (T = 26 hours) |

### II. In-Vitro Release Study: Comparison of Different Concentrations of IPA Gel

The effect of varying the percentage of ingenol mebutate in the gel formulation from 0.1% to 0.01% and 0.001% w/w on the release of the drug substance from the formulation matrix across a synthetic membrane was investigated using Franz diffusion cells under both occluded and non-occluded conditions.

Table 7 below gives a comparison of the flux of ingenol mebutate from the gel as determined from the cumulative amount of ingenol mebutate permeated per unit area per hour for the first 10 hours after application of gel and also after 26 hours.

TABLE 7
Comparison of the flux of ingenol mebutate from the gel at 10 hours
and 26 hours post application, as determined from the cumulative
amount of ingenol mebutate permeated per unit area per hour
(mean ± SEM, n = 3)

Cumulative amount
PEP005 ‘b’ (μg/cm²/h)

| Formulation | 10 h | 26 h |
|---|---|---|
| Nonoccluded | | |
| 0.001% (w/w) PEP005/IPA gel | 0.00 ± 0.00 | 0.03 ± 0.00 |
| 0.01% (w/w) PEP005/IPA gel | 0.89 ± 0.11 | 0.82 ± 0.08 |
| 0.1% (w/w) PEP005/IPA gel | 6.34 ± 0.65 | 4.26 ± 0.26 |
| Occluded | | |
| 0.001% (w/w) PEP005/IPA gel | 0.00 ± 0.00 | 0.04 ± 0.00 |
| 0.01% (w/w) PEP005/IPA gel | 0.26 ± 0.03 | 0.62 ± 0.03 |
| 0.1% (w/w) PEP005/IPA gel | 4.09 ± 0.41 | 7.67 ± 0.52 |

For the first approximately 12 hours after gel application, the release of ingenol mebutate (PEP005) from the 0.1% w/w gel was greater in the non-occluded condition than in the occluded condition. This is possibly because in the case of the non-occluded experiment, the solvent/IPA evaporates over the first 12 hours, thus increasing the concentration of drug in the residual phase of the formulation to an elevated thermodynamic activity state. This effect creates a greater driving force for the diffusion of drug from the gel and through the synthetic membrane compared to the occluded experiment. In the latter case, occlusion should maintain the gel in essentially its original content. The 0.1% PEP005 w/w gel in the occluded experiment showed a classic Fickian response for an infinite dose application, i.e. a steady state of release was achieved after the initial lag time during which time the drug is partitioning from the gel into the membrane. Extrapolation of the linear portion to the time axis gave an estimation of the lag time to be approximately 5 hours. After 12 hours the release of ingenol mebutate from the 0.1% w/w gel in the non-occluded experiment slowed down possibly as a result of the formulation drying up on the membrane. This could reduce the mobility of ingenol mebutate to partition from the formulation into the membrane. This effect did not occur in the non-occluded experiments as the loss of volatiles was minimised due to the donor compartment being occluded.

The 0.01% w/w gel showed a slightly greater overall release in the non-occluded experiment than the occluded experiment, most likely due to reasons described above. At 10 hours the ratio of the release of ingenol mebutate from the 0.01% w/w gel in the non-occluded condition to the release from the occluded condition is approximately 3:1 (see Table 7). By 26 hours, this ratio is reduced to approximately 4:3, indicating that by this time point, the release from the occluded experiment is in line with the release from the non-occluded condition. The release profile for the non-occluded experiment suggests that even with the evaporation of IPA/solvent from this gel, the 0.01% w/w gel was not close to its maximum thermodynamic activity and therefore the total release of ingenol mebutate from the 0.01% gel (nonoccluded) remained much lower than in the 0.1% w/w gel (non-occluded). It was difficult to distinguish between the non-occluded and occluded experiments for the 0.001% w/w el, as the release of ingenol mebutate from this gel was very low. This was probably due to the low concentration of ingenol mebutate in the vehicle such that even the evaporation of solvent did not increase the concentration of drug substance sufficiently close to saturation of the gel.

III. Skin Permeation of Ingenol Mebutate (0.1% w/w) Gel

The primary objective of this study was to determine the rate and extent of permeation of ingenol mebutate (0.1% w/w) gel, including the impurities PEP015 and PEP025, through rat, human and mini-pig skin. The in vitro dermal absorption of ingenol mebutate was determined at 2 application rates; nominally and 150 μg/cm2, through rat, human and mini-pig dermatomed membranes, (which contains the stratum corneum, epidermis and upper layer of dermis). The membranes were occluded throughout the 24-h exposure period. The concentration of PEP015, ingenol mebutate and PEP025 was determined in the receptor fluid and the percentage of the applied dose in each sample and the rate of penetration (ng/cm²/h) was calculated. The permeability co-efficient, Kp (cm/second) was also calculated. Ten membranes from each species were dosed with 150 μL/cm2 of either a 0.1% (1000 μg/mL) formulation or a 0.01% (100 μg/mL) formulation (prepared from the 1000 μg/mL formulation diluted 10-fold with a placebo formulation). Only the low dose samples were analysed as the topical load per unit area at the low dose was more relevant to the potential topical dose in man. Receptor fluid samples were collected at time points following application. The mean absorption parameters from the amounts of PEP015, ingenol mebutate and PEP025 found in the low dose samples were calculated and the data is presented in the Table 8 below. The absorption of ingenol mebutate through the rat, human and mini-pig dermatone membranes following a single application of ingenol mebutate at a nominal dose level of 15 μg/cm² is represented graphically in FIG. 16.

TABLE 8
Mean absorption parameters for ingenol mebutate (PEP005)

| | Administered dose 15 μg/cm² | | | | | |
|---|---|---|---|---|---|---|
| | rat skin | | human skin | | mini-pig skin | |
| Absorption of PEP005 | 2.60 | (±0.65) | 1.93 | (±1.10) | 2.09 | (±0.61) |
| (as percent applied (±SD)) | | | | | | |
| Maximum rate of penetration | 21.6 | (±9.66) | 17.5 | (±10.5) | 18.4 | (±5.27) |
| (ng/cm²/h) (±SD) | | | | | | |
| Lag Time | 4.9 | (±3.1) | 3.7 | (±1.6) | 2.6 | (±0.5) |
| (hours) (±SD) | | | | | | |
| Permeability coefficient | 6.00 × 10⁻⁸ | (±2.68 × 10⁻⁸) | 4.87 × 10⁻⁸ | (±2.92 × 10⁻⁸) | 5.11 × 10⁻⁸ | (±1.46 × 10⁻⁸) |
| (cm/second) (±SD) | | | | | | |

NB: absorption was calculated from the sum of all three isomers found

The main conclusions from the study were:

(i) The mean percentage of ingenol mebutate absorbed (found in the receptor fluid) was 2.60%, 1.93% and 2.09% for rat, human and mini-pig membranes respectively.

(ii) The mean maximum rate of penetration was 21.6 ng/cm²/h in rat membranes, 17.5 ng/cm²/h in human membranes and 18.4 ng/cm²/h in mini-pig membranes at a dose level of 15 μg/cm2.

(iii) The mean rate of penetration over 24 hours was 16.2 ng/cm²/h in rate membranes, 12.1 ng/cm²/h in human membranes and 13.1 ng/cm²/h in mini-pig membranes at 15 μg/cm².

(iv) The calculated mean permeability coefficient was 6.00×10⁻⁸ cm/second in rat membranes, 4.87×10⁻⁸ cm/second in human membranes and 5.11×10⁻⁸ cm/second in mini-pig membranes at 15 μg/cm².

(v) Mean lag times of between 2.6 to 4.9 hours were observed for all species at μg/cm².

(vi) There appeared to be no significant differences of percentage absorption of ingenol mebutate, maximum rate of penetration, lag time and permeability coefficient for rat, mini-pig and human membranes.

### IV. Skin Permeation of [3H]-PEP005 (Ingenol Mebutate)

The rate and extent of absorption of [³H]-ingenol mebutate over a 24 h exposure period following topical application of the gel to Wistar (WI) rat, Sprague Dawley (SD) rat, Gottingen mini-pig and human split-thickness skin was also assessed.

A statistical evaluation of total absorption and dermal delivery of [³H]-ingenol mebutate showed no significant differences for total absorption or dermal delivery of [³H]-ingenol mebutate for the gel for human skin in vitro. Following topical application of [³H]-ingenol mebutate in a gel formulation at ca 0.05% (w/w) to human, mini-pig, SD and WI rat skin in vitro, the absorbed doses of [³H]-ingenol mebutate were 0.21% (0.01 μg equiv./cm²), 0.15% (0.01 μg equiv./cm²), 1.03% (0.05 μg equiv./cm²) and 2.89% (0.12 μg equiv./cm²) respectively. Dermal delivery of [³H]-ingenol mebutate was 0.91% (0.04 μg equiv/cm²), 7.17% (0.31 μg equiv./cm²), 4.66% (0.21 μg equiv./cm²) and 8.39% (0.36 μg equiv./cm²), for each species/strain, respectively. The stratum corneum acted as nature intended with total unabsorbed doses of [³H]-ingenol mebutate recovered from the human, mini-pig, SD and WI rat skins were 101.09%, 93.35%, 94.40% and 90.31% of the applied dose, respectively.

The rank order of total absorption (% applied dose) was WI rat skin >SD rat skin >human skin >mini-pig skin. Compared to human skin the total absorption of [³H]-ingenol mebutate was ca 1.4-fold less for mini-pig skin, ca 4.9-fold greater for SD rat skin and ca 13.8-fold greater for WI rat skin. The rank order of dermal delivery (% applied dose) was WI rat skin >mini-pig skin >SD rat skin >human skin. Compared to human skin dermal delivery of [³H]-ingenol mebutate was ca 5.1-fold greater for SD rat skin, ca 7.9-fold greater for mini-pig skin and ca 9.2-fold greater for WI rat skin. The difference between species/strain for total absorption and dermal delivery (rig equiv./cm²) were also of a similar order of magnitude to those stated above for the percentage data.

The maximum hourly flux was 0.44, 0.80, 2.09, and 6.12 ng equiv./cm²/h for human, mini-pig, SD and WI rat skin, respectively. Maximum flux was achieved at 16, 1, 12 to 14 and 16 h post dose for each species/strain. The flux profile for mini-pig was dissimilar to the other species/strains. For the mini-pig, there was no apparent lag time or steady state flux observed. The other three species/strains had similar flux profiles with lag times of ca 1, 2 and 3 h for the human, SD and WI rat skin, respectively. This was followed by steady state flux from 4-24 h, 6-24 h and 8-24 h, respectively. Steady state flux was 0.39, 2.01 and 5.87 ng equiv./cm²/h for the human, SD and WI rat skin, respectively. Compared to human skin, steady state flux was ca 5.2 and 15.1-fold greater for the SD rat skin and WI rat skin, respectively. These differences with the mini-pig group compared to the other three skin groups may have been attributed to the preparation of the split-thickness skin. Due to the hair follicle depth in mini-pig skin, split-thickness skin was prepared at a depth of ca 1100 pm compared to ca 400 pm for the other skin groups. Therefore, the thicker layer of dermis for the mini-pig skin may have acted as a reservoir for [3H]-PEP005 prior to partitioning into the receptor fluid. A summary of the mean results are provided in Table 9 below.

TABLE 9
Summary of mean absorption results for ingenol mebutate

| | | | Sprague | |
|---|---|---|---|---|
| | | | Dawley | Wistar |
| | Human | Mini-Pig | Rat | Rat |
| Target PEP005 Concentration (% w/w) | 0.05 | 0.05 | 0.05 | 0.05 |
| PEP005 Concentration by Radioactivity (% w/w) | 0.05 | 0.05 | 0.05 | 0.05 |
| Application Level of PEP005 by Radioactivity | 4.34 | 4.34 | 4.42 | 4.26 |
| (μg equiv./cm²) | | | | |
| Dislodgeable Dose (% Applied Dose) | 77.22 | 73.29 | 85.11 | 73.67 |
| Unabsorbed Dose (% Applied Dose) | 101.09 | 93.35 | 94.40 | 90.31 |
| Absorbed Dose (% Applied Dose) | 0.21 | 0.15 | 1.03 | 2.89 |
| Dermal Delivery (% Applied Dose) | 0.91 | 7.17 | 4.66 | 8.39 |
| Mass Balance (% Applied Dose) | 102.00 | 100.52 | 99.05 | 98.70 |
| Dislodgeable Dose (μg equiv./cm²) | 3.35 | 3.17 | 3.76 | 3.14 |
| Unabsorbed Dose (μg equiv./cm²) | 4.39 | 4.05 | 4.17 | 3.85 |
| Absorbed Dose (μg equiv./cm²) | 0.01 | 0.01 | 0.05 | 0.12 |
| Dermal Delivery (μg equiv./cm²) | 0.04 | 0.31 | 0.21 | 0.36 |
| Mass Balance (μg equiv./cm²) | 4.43 | 4.36 | 4.38 | 4.21 |
| Lag Time (h) | 1 | NO | 2 | 3 |
| Steady State Flux (ng equiv./cm²/h) | 0.39 | NO | 2.01 | 5.87 |
| Period of Steady State Flux (h) | 4.24 | NO | 6.24 | 8.24 |

NO = not observed

### Example 7: Methods of Use

### TRADEMARK (Ingenol Mebutate) Gel 0.015% and 0.05%

### Highlights of Prescribing Information

These highlights do not include all the information needed to use TRADEMARK Gel safely and effectively. See full prescribing information for TRADEMARK Gel.

TRADEMARK (ingenol mebutate) Gel, for topical use Initial U.S. Approval: [year]

### Indications and Usage

TRADEMARK Gel is a directed cell death inducer and immune response modifier indicated for the topical treatment of actinic keratosis on the face and scalp and on the trunk and extremities. (1)

### Dosage and Administration

Actinic keratosis on the face and scalp: Apply TRADEMARK Gel 0.015% to the affected area once daily for 3 consecutive days. (2)
Actinic keratosis on the trunk and extremities: Apply TRADEMARK Gel 0.05% to the affected area once daily for 2 consecutive days. (2) TRADEMARK Gel is not for oral, ophthalmic, intravaginal, or anal use. (2)

### Dosage Forms and Strengths

TRADEMARK Gel is a topical gel available in the dosage strengths 0.015% and 0.05%. (3)
The 0.015% dosage strength is for treatment of face and scalp. (3)
The 0.05% dosage strength is for treatment of trunk and extremities. (3)

Contraindications
None. (4)

### Warnings and Precautions

Avoid contact with the eyes. If accidental exposure occurs, eyes should be flushed immediately with large amounts of water and the patient should seek medical care as soon as possible. (5.1)
TRADEMARK Gel must not be ingested. (5.2)
Local skin responses can occur (5.3, 6.1). The localized skin responses are transient and typically resolve within 2 weeks for face and scalp and within 4 weeks for trunk and extremities.

### Adverse Reactions

The most common adverse reactions observed in clinical trials (≧10%) are local skin responses. Local skin responses include erythema, flaking/scaling, crusting, swelling, vesiculation/pustulation, and erosion/ulceration. (6.1) Other reported reactions (≧1%) include application site infection, and periorbital edema. (6.1)

To report SUSPECTED ADVERSE REACTIONS, contact LEO Pharma Inc. at 1-877-494-4536 or FDA at 1-800-FDA-1088 or www.fda.gov/medwatch.

See 17 for PATIENT COUNSELING INFORMATION and FDA approved patient labeling.

### Full Prescribing Information: Contents*

### 1 INDICATIONS AND USAGE

### 2 DOSAGE AND ADMINISTRATION

### 3 DOSAGE FORMS AND STRENGTHS

### 4 CONTRAINDICATIONS

### 5 WARNINGS AND PRECAUTIONS

### 5.1 Eye Exposure

### 5.2 Ingestion

### 5.3 Local Skin Responses

### 5.4 Ultraviolet Light Exposure

### 5.5 Systemic Reactions

### 5.6 Unevaluated Uses

### 6 ADVERSE REACTIONS

### 6.1 Clinical Trial Experience

### 6.2 Clinical Trial Experience—Long-term Safety Follow-up

### 7 DRUG INTERACTIONS

### 8 USE IN SPECIFIC POPULATIONS

### 8.1 Pregnancy

### 8.4 Pediatric Use

### 8.5 Geriatric Use

### 10 OVERDOSAGE

### 11 DESCRIPTION

### 12 CLINICAL PHARMACOLOGY

### 12.1 Mechanism of Action

### 12.2 Pharmacodynamics

### 12.3 Pharmacokinetics

### 13 NONCLINICAL TOXICOLOGY

### 13.1 Carcinogenesis, Mutagenesis, Impairment of Fertility

### 14 CLINICAL STUDIES

### 14.1 Actinic Keratosis of the Face and Scalp

### 14.2 Actinic Keratosis of the Trunk and Extremities

### 16 HOW SUPPLIED/STORAGE AND HANDLING

### 17 PATIENT COUNSELING INFORMATION

### 17.1 Instruction for Use

### 17.2 Local Skin Responses

### 17.3 Systemic Reactions

### 17.4 FDA Approved Patient Labeling

*Sections or subsections omitted from the full prescribing information are not listed.

### Full Prescribing Information

### 1 Indication and Usage

TRADEMARK Gel is indicated for the topical treatment of actinic keratosis on the face and scalp and on the trunk and extremities.

### 2 Dosage and Administration

For topical use only.

For the treatment of actinic keratosis on the face and scalp TRADEMARK Gel 0.015% should be applied to the affected area once daily for 3 consecutive days.

For the treatment of actinic keratosis on the trunk and extremities TRADEMARK Gel 0.05% should be applied to the affected area once daily for 2 consecutive days.

TRADEMARK Gel should be applied to a defined treatment area. A treatment area is defined as one contiguous area of approximately 25 cm² (e.g., 5 cm×5 cm).

The gel from the unit dose tube should be squeezed onto the fingertip and spread evenly over the entire treatment area, allowing it to dry for 15 minutes. One unit dose tube should be used for one treatment area.

Patients should be instructed to wash their hands immediately after applying TRADEMARK Gel.

TRADEMARK Gel is not for oral, ophthalmic, intravaginal, or anal use.

### 3 Dosage Forms and Strengths

TRADEMARK Gel is a topical gel available in unit dose tubes in dosage strengths of 0.015% and 0.05%.

The 0.015% dosage strength is for treatment of face and scalp locations. The 0.05% dosage strength is for treatment of trunk and extremities locations.

### 4 Contraindications

None.

### 5 Warnings and Precautions

### 5.1 Eye Exposure

Avoid contact with the eyes. If accidental exposure occurs, the eyes should be flushed immediately with large amounts of water, and the patient should seek medical care as soon as possible.

### 5.2 Ingestion

TRADEMARK Gel must not be ingested. If accidental ingestion occurs contact your local poison control center.

### 5.3 Local Skin Responses

Mild to moderate local skin responses (LSR) including erythema, flaking/scaling, and crusting can occur after topical application of TRADEMARK Gel [see Adverse Reactions (6)]. These local skin responses have been shown to be positively associated with clinical efficacy. Localized skin responses are transient and typically occur within one day of treatment initiation and peak in intensity up to one week following completion of treatment. Localized skin responses typically resolve within 2 weeks for face and scalp and within 4 weeks for trunk and extremities.

A treatment effect may not be adequately assessed until resolution of local skin responses.

Administration of TRADEMARK Gel is not recommended until the skin is healed from any previous drug or surgical treatment.

### 5.4 Ultraviolet Light Exposure

Studies have been conducted to assess the effects of UV irradiation on the skin following single and multiple applications of ingenol mebutate, 0.01%. Ingenol mebutate did not demonstrate any potential for photoirritation or photoallergy effects. However, excessive exposure to sunlight (including sunlamps and tanning beds) should be avoided or minimized during use of TRADEMARK Gel due to the nature of the disease.

### 5.5 Systemic Reactions

No systemic absorption of TRADEMARK Gel has been detected under maximal use conditions (100 cm² contiguous treatment area treated with 4 unit dose tubes of TRADEMARK Gel 0.05% once daily for 2 consecutive days). [see Clinical Pharmacology 12.3]

### 5.6 Unevaluated Uses

Actinic keratosis (AK) is linked epidemiologically to development of squamous cell carcinoma (SCC), and both conditions share specific gene expression. AK lesions are considered a precursor of SCC, but the effect of TRADEMARK Gel in SCC or prevention of AK developing into SCC has not been studied in long term clinical trials.

The use of TRADEMARK Gel under occlusion has not been investigated for AK lesions.

### 6 Adverse Reactions

Because clinical studies are conducted under widely varying conditions, adverse reaction rates observed in the clinical studies of a drug cannot be directly compared to rates in the clinical studies of another drug and may not reflect the rates observed in practice.

### 6.1 Clinical Trial Experience

The data described below reflect exposure to TRADEMARK Gel or vehicle in 1002 subjects with actinic keratosis treated in four vehicle controlled phase 3 studies. Subjects received field treatment (area of 25 cm²) with TRADEMARK Gel at concentrations of 0.015% or 0.05% or vehicle once daily for 3 or 2 consecutive days, respectively. Adverse reactions were generally mild to moderate in intensity.

TABLE 10
Adverse reactions occurring in ≧1% of subjects
treated with TRADEMARK Gel and at higher frequency than
vehicle

| | TRADEMARK Gel | Vehicle |
|---|---|---|
| Preferred term | (N = 499) | (N = 503) |
| Application site pain | 8.6% | 0.2% |
| Application site pruritus | 8.2% | 0.6% |
| Application site irritation | 2.6% | 0.2% |
| Application site infection | 1.4% | 0.2% |
| Periorbital edema | 1.4% | 0.0% |
| Headache | 1.4% | 1.0% |

In the four vehicle controlled phase 3 studies, local skin responses (erythema, flaking/scaling, crusting, swelling, vesiculation/pustulation, erosion/ulceration) were assessed within the selected treatment area and graded by the investigator on a scale of 0 to 4. A grade of 0 represented no reaction present in the treated area, and a grade of 4 indicated a marked and discernable skin reaction that extended beyond the area treated.

TABLE 11
Local skin responses in the treatment area in TRADEMARK Gel or
vehicle - treated subjects as assessed by the investigator

| Local Skin | | | | | |
|---|---|---|---|---|---|
| Responses | | Face/Scalp | | Trunk/Extremities | |
| Maximum | | TRADEMARK | | TRADEMARK | |
| Score Post | | Gel 0.015% | Vehicle | Gel 0.05% | Vehicle |
| Baseline | Grade | (N = 274) | (N = 271) | (N = 225) | (N = 232) |
| Erythema | 1 | 25 (9%) | 127 (47%) | 31 (14%) | 102 (44%) |
| | 2 | 56 (20%) | 33 (12%) | 94 (42%) | 16 (7%) |
| | 3 | 125 (46%) | 6 (2%) | 61 (27%) | 2 (1%) |
| | 4 | 66 (24%) | 0 (0%) | 34 (15%) | 0 (0%) |
| Flaking/ | 1 | 52 (19%) | 142 (52%) | 52 (23%) | 131 (56%) |
| Scaling | 2 | 91 (33%) | 36 (13%) | 86 (38%) | 15 (6%) |
| | 3 | 98 (36%) | 4 (1%) | 66 (29%) | 3 (1%) |
| | 4 | 25 (9%) | 0 (0%) | 18 (8%) | 0 (0%) |
| Crusting | 1 | 85 (31%) | 47 (17%) | 105 (47%) | 38 (16%) |
| | 2 | 64 (23%) | 5 (2%) | 39 (17%) | 4 (2%) |
| | 3 | 64 (24%) | 0 (0%) | 23 (10%) | 2 (1%) |
| | 4 | 16 (6%) | 0 (0%) | 8 (4%) | 0 (0%) |
| Swelling | 1 | 88 (32%) | 12 (4%) | 65 (29%) | 13 (6%) |
| | 2 | 67 (24%) | 2 (1%) | 51 (23%) | 0 (0%) |
| | 3 | 48 (18%) | 0 (0%) | 20 (9%) | 0 (0%) |
| | 4 | 14 (5%) | 0 (0%) | 7 (3%) | 0 (0%) |
| Vesiculation/ | 1 | 36 (13%) | 1 (0%) | 46 (20%) | 1 (0%) |
| Pustulation | 2 | 53 (19%) | 0 (0%) | 30 (13%) | 1 (0%) |
| | 3 | 50 (18%) | 0 (0%) | 19 (8%) | 0 (0%) |
| | 4 | 15 (5%) | 0 (0%) | 3 (1%) | 0 (0%) |
| Erosion/ | 1 | 55 (20%) | 4 (1%) | 37 (16%) | 6 (3%) |
| Ulceration | 2 | 26 (10%) | 0 (0%) | 15 (7%) | 0 (0%) |
| | 3 | 5 (2%) | 0 (0%) | 4 (2%) | 0 (0%) |
| | 4 | 1 (0%) | 0 (0%) | 2 (1%) | 0 (0%) |

For both treatment locations (face/scalp and trunk/extremities), erythema and flaking/scaling were the most common LSRs, followed by crusting and swelling in patients treated with TRADEMARK Gel. The local skin responses are transient and typically occur within one day of treatment initiation and peak in intensity up to one week following completion of treatment. Local skin responses typically resolve within 2 weeks for areas treated on the face and scalp and within 4 weeks for areas treated on the trunk and extremities.

Other less common adverse reactions (less than 1% but more than 0.5%) were, in decreasing order: application site parasthesia, back injury and eyelid edema.

### 6.2 Clinical Trial Experience—Long-Term Safety Follow-Up

Three prospective, observational long-term follow-up studies were conducted to evaluate recurrence of actinic keratosis lesions and safety in subjects receiving treatment with TRADEMARK Gel on the face, scalp, trunk or extremities. Only those subjects who achieved complete clearance in the treated area at the end of the phase 3 studies (Day 57) were eligible for long term follow-up. Subjects were followed every 3 months for up to 12 months.

A total of 198 subjects (184 treated with TRADEMARK Gel and 14 treated with vehicle) enrolled in the long-term follow-up studies. Of these, 117 subjects had been treated on the face and scalp and 81 subjects had been treated on the trunk and extremities. X % of the patients, across all three studies, completed 12 months of follow-up. X SAEs were reported for X patients across all three studies during the 12 month follow-up. X adverse events were reported.

### 7 Drug Interactions

TRADEMARK Gel is not absorbed systemically; therefore no formal systemic drug interaction studies have been performed. [see Clinical Pharmacology (12.3).

### 8 Use in Specific Populations

### 8.1 Pregnancy

### Pregnancy Category B

Based on a lack of systemic exposure following dermal application there is no appreciable reproductive risk to humans receiving therapeutic exposure to TRADEMARK Gel. There are, however, no adequate and well-controlled studies in pregnant women.

Because animal reproduction studies are not always predictive of human response, this drug should be used during pregnancy only if clearly needed.

Teratogenicity studies with ingenol mebutate were performed by the intravenous route in rats and rabbits.

In rats, ingenol mebutate was not associated with fetal developmental effects at doses up to 5 μg/kg/day (30 μg/m²/day). In rabbits, maternal toxicity (increased breathing) was observed at doses ≧1 μg/kg/day (12 μg/m²/day), and a slight increase in the incidence of incompletely ossified cervical vertebral arches was observed at doses ≧2 μg/kg/day (24 μg/m²/day). A dosage of 4 μg/kg/day (48 μg/m²/day) resulted in a slight increase in embryonic deaths, an increase in jugular fusion to the maxilla and a variation in the origin of arteries arising from the aortic arch.

The fetal NOAEL was 1 μg/kg/day (12 μg/m²/day).

All embryo-fetal findings were marginal and did not reach statistical significance.

### 8.4 Pediatric Use

Actinic keratosis is not a condition generally seen within the pediatric population.

The safety and efficacy of TRADEMARK Gel for actinic keratosis in patients less than 18 years of age have not been established.

### 8.5 Geriatric Use

Of the 1165 subjects treated with TRADEMARK Gel in the clinical studies, 656 subjects (56.3%) were 65 years and older and, 241 subjects (20.7%) were 75 years and older. No overall differences in safety or effectiveness were observed between these subjects and younger subjects.

### 10 Overdosage

There has been no experience of overdose with TRADEMARK Gel. Exposure of TRADEMARK Gel 0.05% applied daily for two consecutive days to areas of skin up to 100 cm² for the treatment of actinic keratosis did not change the safety profile. [see Clinical Pharmacology (12.3)]

No incidents of accidental oral ingestion have been reported.

### 11 Description

TRADEMARK Gel is a clear colorless gel and is intended for topical administration.

It contains the active substance ingenol mebutate.

Ingenol mebutate has the chemical name;

2-Butenoic acid, 2-methyl-, (1aR,2S,5R,5aS,6S,8aS,9R,10aR)-1a,2,5,5a,6,9,10,10a-octahydro-5,5a-dihydroxy-4-(hydroxymethyl)-1,1,7,9-tetramethyl-11-oxo-1H-2,8a-methanocyclopenta[a]cyclopropa[e]cyclodecen-6-yl ester, (2Z)—

or

(1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H 2,8a-methanocyclopenta[a]cyclopropa[e]cyclodecen-6-yl (2Z) 2 methylbut-2-enoate.

Ingenol mebutate has the molecular formula C₂₅H₃₄O₆ and a molecular weight of 430.5. Its structural formula is:

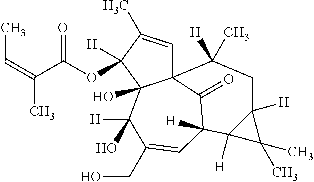 [SMILES: C/C=C(/C)C(=O)O[C@H]1C(C)=CC23C(=O)[C@@H](C=C(CO)[C@@H](O)[C@]12O)C1C(C[C@H]3C)C1(C)C]

Ingenol mebutate is a white to pale yellow crystalline powder.

TRADEMARK Gel 0.015% and 0.05% contains per gram 150 mcg and 500 mcg of ingenol mebutate, respectively in a gel base of isopropyl alcohol, hydroxyethyl cellulose, citric acid monohydrate, sodium citrate dihydrate, benzyl alcohol and purified water.

### 12 Clinical Pharmacology

### 12.1 Mechanism of Action

The mechanism of action in AK is not fully understood. In vivo and in vitro models have shown a dual mechanism of action for the effects of ingenol mebutate: 1) induction of local lesional cell death and 2) promoting an inflammatory response characterized as infiltration of neutrophils and other immunocompetent cells.

### 12.2 Pharmacodynamics

Ingenol mebutate is a pleiotropic effector that exerts a direct cytotoxic effect on tumor cells and modulates Protein Kinase C (PKC) isoforms. At high concentrations (e.g., 100 μg/mL), ingenol mebutate induces mitochondrial swelling and loss of cell membrane integrity leading to cell death; at subclinical concentrations (e.g., 10 to 100 ng/mL), ingenol mebutate stimulates PKC dependent activation of human endothelial cells to support neutrophil adhesion in vitro.

Exposure of isolated human keratinocytes to subclinical concentrations of ingenol mebutate in vitro has shown cytokine release and specific PKC-mediated neutrophil activation, and its application to mouse skin in subclinical concentrations in vivo induced the release of inflammatory mediators IL-8/MIP-2 and TNF-α resulting in neutrophil recruitment and activation.

In mouse xenograft squamous cell carcinoma and melanoma tumor models, topical ingenol mebutate removes tumors and prevents tumor recurrence by a dual mechanism of action 1) induction of local lesional cell death and 2) promoting an inflammatory response characterized as infiltration of neutrophils and other immunocompetent cells.

After 3 weeks treatment in the squamous cell carcinoma xenograft model, ingenol mebutate-treated mouse skin was similar to untreated skin in elasticity and by 2 to 3 months had little scarring or erythema demonstrating a favorable cosmetic effect.

With intratumoral injection in mouse xenograft models, ingenol mebutate stimulated a tumor-specific CD8+T cell response with anti-tumor activity against distant secondary tumors.

The potential for off-target activity was investigated in various in vitro assays and ingenol mebutate did not inhibit or stimulate receptors and enzymes.

### 12.3 Pharmacokinetics

The systemic pharmacokinetic profile of ingenol mebutate and its metabolites has not been characterized in humans due to an absence of quantifiable whole blood levels following topical administration. No systemic absorption was detected at or above the lower limit of detection (0.1 ng/mL) when TRADEMARK Gel 0.05% from 4 unit dose tubes was applied to an area of 100 cm² of the dorsal forearm in AK patients once daily for two consecutive days.

In vitro study results demonstrate that ingenol mebutate does not inhibit or induce human cytochrome P450 isoforms.

### 13 Nonclinical Toxicology

### 13.1 Carcinogenesis, Mutagenesis, Impairment of Fertility

Carcinogenic evaluations of ingenol mebutate have not been conducted. Ingenol mebutate was not mutagenic in an in vitro Ames test, mouse lymphoma assay, and in vivo rat micronucleus test. Ingenol mebutate was positive in the Syrian hamster embryo (SHE) cell transformation assay at concentrations 0.1 μg/mL.

No fertility studies have been performed with ingenol mebutate.

### 14 Clinical Studies

### 14.1 Actinic Keratosis of the Face and Scalp

In two double-blind, vehicle-controlled, clinical studies, 547 adult subjects with AK on the face or scalp were randomized to treatment with either TRADEMARK Gel 0.015% or vehicle gel for 3 consecutive days. Subjects then continued in the study for an 8 week follow-up period during which they returned for clinical observations and safety monitoring. The studies enrolled subjects with 4 to 8 clinically typical, visible, discrete AK lesions within a 25 cm² contiguous treatment area. Hypertrophic and hyperkeratotic lesions were excluded from treatment. On each scheduled dosing day, the study gel was applied to the entire treatment area. A total of 536 subjects (98%) completed these studies. Study subjects ranged from 34 to 89 years of age (mean 64.1 years) and 94.1% had Fitzpatrick skin type I, II, or III.

Efficacy was assessed at Day 57. Complete clearance rate was defined as the proportion of subjects with no (zero) clinically visible AK lesions in the treatment area. Partial clearance rate was defined as the percentage of subjects in whom 75% or more of the number of baseline AK lesions were cleared. Median percent (%) reduction in AK lesions compared to baseline was also calculated.

TABLE 12
Rates of subjects with complete and partial clearance and
percent (%) reduction on face and scalp

| | Study 1 | | Study 2 | |
|---|---|---|---|---|
| | TRADEMARK | | TRADEMARK | |
| | Gel 0.015% | Vehicle | Gel 0.015% | Vehicle |
| | (n = 135) | (n = 134) | (n = 142) | (n = 136) |
| Complete | 37.0%^{a} | 2.2% | 47.2%ₐ | 5.1% |
| Clearance | | | | |
| Rate | | | | |
| Partial | 60.0%ₐ | 6.7% | 67.6%ₐ | 8.1% |
| Clearance | | | | |
| Rate (≧75%) | | | | |
| Median % | 83.3% | 0.0% | 86.6% | 0.0% |
| Reduction | | | | |

^{a}p < 0.001; compared to vehicle by Cochran-Mantel-Haenszel stratifying on analysis site

One prospective, observational long-term follow-up study was conducted to evaluate recurrence of actinic keratosis lesions and safety in subjects receiving treatment with TRADEMARK Gel on the face and scalp. Only those subjects who achieved complete clearance in the treated area at the end of the phase 3 studies (Day 57) were eligible for long term follow-up. Subjects were followed every 3 months for up to 12 months.

Recurrence was defined as any identified AK lesion in the previously treated area for patients who achieved complete clearance at Day 57 in the previous phase 3 study [see Adverse Reactions (6.2)].

TABLE 13
Rate of recurrence of actinic keratosis lesions on face and scalp

| | Number of | Recurrence | Lesion Based |
|---|---|---|---|
| | subjects | Rate* (%) | Recurrence Rate |
| Study 3 | 117 | X | X |

*The recurrence rate is the Kaplan-Meier “failure” estimate at the target study date of the visit expressed as a percentage.

### 14.2 Actinic Keratosis of the Trunk and Extremities

In two double-blind, vehicle-controlled clinical studies, 458 adult subjects with AK on the trunk or extremities were randomized to treatment with either TRADEMARK Gel 0.05% or vehicle gel for 2 consecutive days. Subjects then continued in the study for an 8 week follow-up period during which they returned for clinical observations and safety monitoring. The studies enrolled subjects with 4 to 8 clinically typical, visible, discrete AK lesions within a 25 cm² contiguous treatment area. Hypertrophic and hyperkeratotic lesions were excluded from treatment. On each scheduled dosing day, the study gel was applied to the entire treatment area. A total of 447 subjects (97.6%) completed these studies. Study subjects ranged from 34 to 89 years of age (mean 66.2 years) and 93.9% had Fitzpatrick skin type I, II, or III.

Efficacy was assessed at Day 57. Complete clearance rate was defined as the proportion of subjects with no (zero) clinically visible AK lesions in the treatment area. The partial clearance rate was defined as the percentage of subjects in whom 75% or more of the number of baseline AK lesions were cleared. Median percent (%) reduction in AK lesions compared to baseline was also calculated.

TABLE 14
Rates of subjects with complete and partial
clearance and percent (%) reduction on trunk and

| | Study 4 | | Study 5 | |
|---|---|---|---|---|
| | TRADEMARK | | TRADEMARK | |
| | Gel 0.05% | Vehicle | Gel 0.05% | Vehicle |
| | (n = 126) | (n = 129) | (n = 100) | (n = 103) |
| Complete | 27.8%ₐ | 4.7% | 42.0%ₐ | 4.9% |
| Clearance | | | | |
| Rate | | | | |
| Partial | 44.4%ₐ | 7.0% | 55.0%ₐ | 6.8% |
| Clearance | | | | |
| Rate (≧75%) | | | | |
| Median % | 69.1% | 0.0% | 75.0% | 0.0% |
| Reduction | | | | |

^{a}p < 0.001; compared to vehicle by Cochran-Mantel-Haenszel stratifying on analysis site

Two prospective, observational long-term follow-up studies were conducted to evaluate recurrence of actinic keratosis lesions and safety in subjects receiving treatment with TRADEMARK Gel on the trunk and extremities. Only those subjects who achieved complete clearance in the treated area at the end of previous phase 3 studies (Day 57) were eligible for long term follow-up. Subjects were followed every 3 months for up to 12 months.

Recurrence was defined as any identified AK lesion in the previously treated area for patients who achieved complete clearance at Day 57 in a previous phase 3 study. [see Adverse Reactions (6.2)]

TABLE 15
Rate of recurrence of actinic keratosis lesions on trunk and
extremities

| | | Recurrence Rate* | Lesion Based |
|---|---|---|---|
| | Number of | (%) | Recurrence Rate |
| | subjects | 12 months | 12 months |
| Study 6²⁰ | 38 | X | X |
| Study 7²¹ | 43 | X | X |

*The recurrence rate is the Kaplan-Meier “failure” estimate at the target study date of the visit expressed as a percentage.

### 16 how Supplied/Storage and Handling

TRADEMARK is supplied in unit dose laminate tubes containing 0.47 g of TRADEMARK Gel.

TRADEMARK Gel is available in 2 dosage strengths: 0.015% and 0.05%.

| | Number of unit dose | |
|---|---|---|
| Dosage Strength | tubes per carton | NDC# |
| 0.015% | 3 | xxxxx-xxx-xx |
| 0.05% | 2 | xxxxx-xxx-xx |

TRADEMARK Gel should be stored in a refrigerator 36° F.-46° F. (2° C.-8° C.); excursions permitted between 32° F.-59° F. (0° C.-15° C.). Avoid freezing.

Keep Out of Reach of Children. Rx Only.

### 17 Patient Counseling Information

### 17.1 Instruction for Use

TRADEMARK Gel should be used as directed by a physician. Dosing is once a day for 3 consecutive days for treatment of face and scalp and once a day for 2 consecutive days for treatment of trunk and extremities. [see Dosage and Administration (2)].

TRADEMARK Gel is for external use only. Contact with the eyes should be avoided. [see Dosage and Administration (2) and Warnings and Precautions (5.1)].

The treatment area should not be bandaged or covered with other closed dressings. [see Warnings and Precautions (5.6)]

Patients should wash their hands immediately after applying TRADEMARK Gel.

It is recommended that patients avoid excessive exposure to sunlight (including sunlamps and tanning beds) while using TRADEMARK Gel. [see Warnings and Precautions (5.4)]

### 17.2 Local Skin Responses

Patients should be informed that treatment with TRADEMARK Gel may lead to local skin responses. This includes erythema, flaking/scaling, crusting, swelling, vesiculation/pustulation, and erosion/ulceration. These reactions can range from mild to moderate in intensity and may extend beyond the application site onto the surrounding skin. Localized skin responses typically occur within one day after treatment initiation and resolve within 2 weeks for face and scalp and within 4 weeks for trunk and extremities.

Patients may also experience application site reactions such as pruritus and pain. [see Adverse Reactions (6.1)

### 17.3 Systemic Reactions

Because there is no systemic absorption of TRADEMARK Gel, systemic reaction to TRADEMARK Gel is unlikely. [see Clinical Pharmacology (12.3)].

### 17.4 FDA Approved Patient Labeling

### Patient Information

### TRADEMARK (Ingenol Mebutate) Gel, 0.015% and 0.05%

IMPORTANT: For Use on Skin Only. Not for Oral, Eye, Vaginal, or Anal Use.

Read the Patient Information leaflet that comes with TRADEMARK Gel before you start using it and each time you get a refill. There may be new information. This leaflet does not take the place of talking with your healthcare provider about your medical condition or treatment. If you do not understand the information, or have any questions about TRADEMARK Gel, talk with your healthcare provider or pharmacist.

### What is TRADEMARK Gel?

TRADEMARK Gel is a medicine for use on the skin to treat actinic keratosis in adults. The condition actinic keratosis is also known as solar keratosis since it is most often caused by ultraviolet rays from too much sun exposure.

Who should not Use TRADEMARK Gel?
TRADEMARK Gel has not been studied in children less than 18 years for actinic keratosis. Actinic keratosis does not usually occur in children.

What should I tell my doctor before using TRADEMARK Gel?
Tell your healthcare provider about all your medical conditions, and about all the medicines you take including prescription and non-prescription medicines, vitamins and herbal supplements. It is not known if TRADEMARK Gel and other medicines can affect each other.
In particular, tell your healthcare provider if you have had other treatments for actinic keratosis, or are using other medicines applied to the skin.
   How should I use TRADEMARK?
Use TRADEMARK Gel exactly as prescribed by your healthcare provider.
TRADEMARK Gel 0.015% is used once a day for 3 days in a row for treating face and scalp.
TRADEMARK Gel 0.05% is used once a day for 2 days in a row for the treatment of body, arms and legs.
Use TRADEMARK Gel only on the area to be treated as described by your healthcare provider.
Do not use TRADEMARK Gel longer than prescribed. Using too much TRADEMARK Gel can increase your risk of having a severe skin reaction or other side effects.
Do not use TRADEMARK Gel until your skin has healed from other treatments.

### Applying TRADEMARK Gel

Remove cap from tube just before use.
Squeeze content of one tube of TRADEMARK Gel onto fingertip.
The content of one tube will cover an area of approximately 2 inches by 2 inches.
TRADEMARK Gel should be smoothed gently onto the skin.
Avoid touching the treatment area after application; and allow the area to dry for 15 minutes.
After applying TRADEMARK Gel, immediately wash your hands well.
Do not apply immediately after taking a shower or less than 2 hours before bedtime.
Do not wash the areas where you applied TRADEMARK Gel for at least 6 hours after you apply it.
Do not allow anyone or any pets to come in physical contact with the treatment area for a period of 6 hours after applying TRADEMARK Gel
Discard the tube after use.
   What should I Avoid while Using TRADEMARK Gel?
Do not get TRADEMARK Gel in your eyes. Do not touch your eyes while you are applying TRADEMARK Gel and wash your hands well after applying it. Irritation may occur if you get TRADEMARK Gel in your eyes. If you accidentally get TRADEMARK Gel in your eyes, flush the eye region thoroughly with water and seek medical care immediately.
Do not get TRADEMARK Gel in your mouth. If you accidentally get TRADEMARK Gel in your mouth, contact your local poison control center.
Do not cover the treated site with bandages or other closed dressings.
Do not use sunlamps and tanning beds and avoid sunlight as much as possible during treatment with TRADEMARK Gel.

### What are the Possible Side Effects of TRADEMARK Gel?

The most common side effects with TRADEMARK Gel are skin reactions at the treatment site including:
redness
flaking
itching
scabbing and crusting
swelling
blister, or ulcer
skin that becomes hard or thickened
skin peeling
changes in skin color
pain at the treatment area

These are not all the side effects of TRADEMARK Gel. For more information, ask your healthcare provider or pharmacist.

You may have a severe skin reaction if you use too much TRADEMARK Gel or use it the wrong way. Skin reactions typically resolve within 2-4 weeks.

If you have questions regarding treatment or skin reactions, please talk with your healthcare provider.

You may report side effects to LEO Pharma Inc. at 1-877-494-4536 or to FDA at 1 800 FDA-1088 or www.fda.gov/medwatch.

### How do I Store TRADEMARK Gel?

Store TRADEMARK Gel in a refrigerator 36° F.-46° F. (2° C.-8° C.). Do not freeze.
TRADEMARK Gel has an expiration date (exp) marked on the tube. Do not use the gel after this date.
Used TRADEMARK Gel tubes should be discarded in household trash in a manner that prevents accidental application or accidental ingestion by children or pets.
Keep TRADEMARK Gel and all medicines out of the reach of children and pets.
   General Information about TRADEMARK Gel

Medicines are sometimes prescribed for conditions that are not mentioned in Patient Information leaflets. Do not use TRADEMARK Gel for a condition for which it was not prescribed. Do not give TRADEMARK Gel to other people, even if they have the same symptoms you have. It may harm them. This Patient Information leaflet summarizes the most important information about TRADEMARK Gel. If you would like more information, talk with your healthcare provider. You can ask your pharmacist or healthcare provider for information about TRADEMARK Gel that is written for healthcare professionals. Ask them to explain anything that you do not understand. If you have other questions about TRADEMARK Gel or for additional information, visit www.XXXXXXXX.com or call 1-877-494-4536.

### What are the Ingredients in TRADEMARK Gel?

Active Ingredient: ingenol mebutate.

Inactive ingredients: isopropyl alcohol, hydroxyethyl cellulose, citric acid monohydrate, sodium citrate dihydrate, benzyl alcohol and purified water.

### Rx Only

The disclosures of the patents, patent documents, articles, abstracts and other publications cited herein are incorporated herein by reference in their entireties as if each were individually incorporated. In case of conflict, the present specification, including definitions, shall control. Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1.-44. (canceled)

45. A topical method of treating a subject, who is diagnosed with actinic keratosis on the subject's face or scalp, with a gel formulated with about 0.015% ingenol-3-angelate, by weight, wherein localized skin responses caused by the gel are resolved within about two weeks following said topical method, said topical method comprising:
(a) squeezing a tube containing about 0.47 grams of the gel to dispense the gel onto a fingertip of the subject;
(b) applying the gel dispensed onto the fingertip to a treatment area on the subject's skin located on the subject's face or scalp, wherein the actinic keratosis is located, to topically treat the actinic keratosis in the treatment area, and wherein the treatment area is defined as one contiguous area of approximately 25 cm²;
(c) allowing the applied gel on the treatment area to dry for about 15 minutes;
(d) repeating said steps (a), (b) and (c) once daily for three consecutive days to treat the actinic keratosis on the subject's face or scalp;
(e) observing localized skin responses caused by the gel within about one day following the first day of said gel treatment;
(f) observing peak intensity of localized skin responses up to about one week following the 3rd day of said gel treatment; and
(g) resolving localized skin responses within about two weeks following the 3rd day of said gel treatment.

46.-59. (canceled)

60. A method of topically treating actinic keratosis on a subject's face or scalp using a pharmaceutical gel formulation comprising about 0.015% ingenol-3-angelate, by weight, said topical method comprising:
(a) squeezing a tube containing about 0.47 grams of the pharmaceutical gel formulation to dispense at least some of the pharmaceutical gel formulation onto a fingertip of the subject;
(b) applying the dispensed pharmaceutical gel formulation to an actinic keratosis treatment area on the subject's face or scalp, wherein the actinic keratosis treatment area is one contiguous area of approximately 25 cm²;
(c) allowing the applied pharmaceutical gel formulation on the treatment area to dry for about 15 minutes;
(d) repeating said steps (a), (b) and (c) once daily for three consecutive days to treat actinic keratosis on the subject's face or scalp;
(e) observing localized skin responses, caused by applying the pharmaceutical gel formulation, within about one day following the first day of said gel treatment;
(f) observing peak intensity of said localized skin responses, up to about one week following the 3rd day of said gel treatment; and
wherein said localized skin responses are resolved within about two weeks following the 3rd day of said topical treatment.

61. A method of topically treating actinic keratosis on a subject's trunk and extremities using a pharmaceutical gel formulation comprising about 0.05% ingenol-3-angelate, by weight, said topical method comprising:
(a) applying the pharmaceutical gel formulation to an actinic keratosis treatment area on the subject's trunk or extremities, wherein the actinic keratosis treatment area is one contiguous area of approximately 25 cm²; and
(b) repeating said step (a) once daily for two consecutive days to treat actinic keratosis on the subject's trunk or extremities.

62. The method of claim 61, wherein said pharmaceutical gel formulation is provided in a tube containing about 0.47 grams of the pharmaceutical gel formulation.

63. The method of claim 62, further comprising squeezing the tube containing the pharmaceutical gel formulation to dispense at least some of the pharmaceutical gel formulation onto a fingertip of the subject for application to the actinic keratosis treatment area.

64. The method of claim 61, further comprising observing localized skin responses, caused by application of the pharmaceutical gel formulation, within about one day following the first day of said gel treatment.

65. The method of claim 64, further comprising observing peak intensity of said localized skin responses, up to about one week following the second day of said gel treatment, and
wherein said localized skin responses are resolved within about four weeks following the second day of said topical treatment.

66. A method of topically treating actinic keratosis on a subject's trunk and extremities using a pharmaceutical gel formulation comprising about 0.05% ingenol-3-angelate, by weight, said topical method comprising:
(a) squeezing a tube containing about 0.47 grams of the pharmaceutical gel formulation to dispense at least some of the pharmaceutical gel formulation onto a fingertip of the subject;
(b) applying the dispensed pharmaceutical gel formulation to an actinic keratosis treatment area on the subject's trunk or extremities, wherein the actinic keratosis treatment area is one contiguous area of approximately 25 cm²;
(c) allowing the applied pharmaceutical gel formulation on the treatment area to dry for about 15 minutes;
(d) repeating said steps (a), (b) and (c) once daily for two consecutive days to treat actinic keratosis on the subject's trunk or extremities;
(e) observing localized skin responses, caused by application of the pharmaceutical gel formulation, within about one day following the first day of said gel treatment;
(f) observing peak intensity of said localized skin responses, up to about one week following the second day of said gel treatment; and
wherein said localized skin responses are resolved within about four weeks following the second day of said topical treatment.
